(19) United States
(12) Patent Application Publication
(10) Pub. No.: US 2021/0333185 A1
(43) Pub. Date: Oct. 28, 2021

(54) TESTING OF AGRICULTURAL PRODUCTS VOLATILES TO PREDICT QUALITY USING MACHINE LEARNING

(71) Applicant: Apeel Technology, Inc., Goleta, CA (US)

(72) Inventors: Taylor Hayward, Santa Barbara, CA (US); Allison Ferranti, Santa Barbara, CA (US); Elaine Kirschke, Goleta, CA (US); Zoe Friedberg, Goleta, CA (US); Matthew Lee, Goleta, CA (US)

(21) Appl. No.: 17/241,576

(22) Filed: Apr. 27, 2021

Related U.S. Application Data
(60) Provisional application No. 63/016,074, filed on Apr. 27, 2020.

(51) Int. Cl. G01N 7/18; G01N 7/04; G06Q 10/08

(52) U.S. Cl. CPC G01N 7/18; G06Q 10/087; G01N 7/04

(57) ABSTRACT

This disclosure is directed to systems and methods for assessing quality characteristics of food items based on analyzing volatiles outgassed by them. The quality characteristics can include presence of infection, ripeness stage, flavor, taste, and smell. Determining quality characteristics can be advantageous to make supply chain modifications that optimize on quality and reduce food-based waste. A tube having a sorbent material can be placed in an environment containing the food items. Volatiles outgassed by the food items can collect on the sorbent material. A computing system can receive the volatiles presence and concentration data and can apply a machine learning model to the data to determine quality characteristics of the food items. The model can be trained using human observations of quality characteristics, historic supply chain information, and processed volatiles data associated with other food items, wherein the other food items are a same type as the food items.

### CROSS-REFERENCE TO RELATED APPLICATIONS

This application claims priority to U.S. Provisional Application Ser. No. 63/016,074, filed on Apr. 27, 2020, the disclosure of which is incorporated by reference in its entirety.

### TECHNICAL FIELD

This document describes devices, systems, and methods related to non-destructively testing agricultural products to assess quality of these products.

### BACKGROUND

Quality of agricultural products, such as fruits and produce, can influence consumers' decisions whether to buy and/or eat those products. Thus, consumer acceptance of agricultural products can be largely impacted by flavor, ripeness, and manifestation of infection exhibited by those products. Agricultural products that are overripe or show signs of infection can often be rejected by consumers, resulting in agricultural product-based waste.

Ripening different agricultural products can also be challenging based on year and season, since the products can have variable initial characteristics, such as dry matter content in avocados. Such variable initial characteristics can result in various times for the products to ripen and/or exhibit preferred quality characteristics. Moreover, in some storage rooms, different agricultural products can ripen differently. The different products may require different storage conditions to ripen and/or exhibit preferred quality characteristics. These products can be tested for ripeness and/or quality characteristics using destructive techniques, such as scraping, puncturing, or slicing the products, at predetermined times during ripening.

Consumers and relevant stakeholders in a supply chain can test the agricultural products' quality using destructive techniques, such as squeezing the product or puncturing an outer skin of the product. Sometimes, the destructive techniques may cause the product to no longer be suitable for purchase and consumption by consumers. Sometimes, the destructive techniques may be performed too late in the supply chain. Thus, the destructive techniques can cause additional agricultural product-based waste.

### SUMMARY

The document generally describes systems, methods, and techniques for non-destructively assessing quality and/or flavor characteristics of agricultural products (e.g., produce, fruit, etc.) before they are distributed to consumers. The disclosed technology can allow for implementation of proactive strategies to mitigate infection and/or control ripening of such agricultural products. Such strategies can be useful to ensure that distributed agricultural products are of high quality when acquired by consumers, thereby, reducing agricultural product-based waste generated by consumer or distribution center rejection. The disclosed technology can provide for determining and/or predicting ripeness, flavor, vascular browning, and stem rot of different agricultural products. The disclosed technology can provide for automatically measuring volatiles that outgas from agricultural products, which can be correlated to different stages in a ripening process and quality characteristics. The disclosed technology can be used as a continuous measure of real-time ripening of agricultural products, thereby ensuring consistent quality products to end consumers. Making such determinations and predictions as early as possible in a supply chain lifecycle can be advantageous to ensure that the agricultural products are appropriately provided to consumers to avoid or mitigate product-based waste.

The disclosed technology can include an apparatus for collecting volatiles of an agricultural product. The apparatus can include a tube having a sorbent material. The tube can be placed in cold storage, a controlled atmosphere, or another type of container having one or more agricultural products. Volatiles from the agricultural product can collect on the sorbent material of the tube. A time to collect the volatiles can be adjusted depending on detection limits of compounds of interest. For example, a longer collection time can achieve lower detection limits as the volatiles are trapped and concentrated on the sorbent material. The tube can then be passed through a gas chromatograph machine to identify the volatiles collected on the sorbent material and to classify the volatiles with quality characteristics of the agricultural product.

In some implementations, volatiles can be collected by drawing air into a gas chromatograph via a pump. Volatiles can also be collected by pushing air into the gas chromatograph by use of a pneumatic control module (PCM). In other words, the gas chromatograph can be purged with nitrogen to force volatiles into a loop where the volatiles can be collected. A gas sampling valve can be used to divert the air into an inlet of the gas chromatograph, where a sorbent material (e.g., Tennax) can be packed into a gas chromatograph liner or other similar tube, as described herein. The volatiles can therefore be trapped and collected on the sorbent material in the inlet. In some implementations, the inlet can be held at a room temperature. A multi-mode inlet can also be used to ramp a temperature of the inlet, thereby desorbing the trapped volatiles form the sorbent material.

The volatile classifications can be used to determine one or more supply chain modifications before the agricultural product is routed towards a destination (e.g., grocery stores, food processing plants, restaurants, etc.). Moreover, the volatiles can be classified using a trained machine learning model. The model can be trained using different input data, such as human observations, historic data associated with the agricultural product, and sensed volatiles associated with the agricultural product. The model can therefore map volatiles data to different product features to determine and predict quality characteristics of the product during run-time.

One or more embodiments described herein can include a system for determining quality characteristics of food items, the system having a tube that can be placed in an environment containing one or more food items, the tube defining a fluid passageway, and a sorbent material positioned within the fluid passageway defined by the tube. The tube with the sorbent material can be positioned near one or more food items to collect one or more volatiles that are outgassed by the one or more food items and to be processed by a gas chromatograph machine. The gas chromatograph machine can receive the tube with the sorbent material after the volatiles collected on the sorbent material and can generate output data that indicates presence and concentrations of the one or more volatiles desorbed from the sorbent material. The system can also include a computing system that can receive the output data generated by the gas chromatograph machine and determine, based on applying a machine learning model to the output data, a quality characteristic of the one or more food items. The machine learning model can be trained using (i) human observations of one or more quality characteristics of other food items, (ii) historic supply chain information of the other food items, and (iii) processed volatiles data associated with the other food items. The other food items can be a same type as the one or more food items.

In some implementations, one or more embodiments can optionally include one or more of the following features. For example, the sorbent material can include 0.07 g to 0.14 g of a porous polymer.

As another example, the system can also include an enclosure containing the one or more food items. The enclosure can define a first aperture that can provide fluid communication between gas contained within the enclosure and an ambient environment outside of the enclosure. The tube can be positioned within the first aperture and, when positioned in the aperture, can provide a fluid passageway between the gas contained in the enclosure and the ambient environment.

In some implementations, the enclosure can define a second aperture between the ambient environment and gas contained within the enclosure and the system can also include one or more fans that are in fluid communication with the second aperture and that can provide a positive pressure flow of gas from the ambient environment into the enclosure that directs the volatiles outgassed by the one or more food items towards and through the tube containing the sorbent material.

In some implementations, the enclosure can define a second aperture between the ambient environment and gas contained within the enclosure, and the system can also include a pressurized gas supply that can be in fluid communication with the second aperture and that can provide a positive pressure flow of gas from the pressurized gas supply into the enclosure that directs the volatiles outgassed by the one or more food items towards and through the tube containing the sorbent material.

The gas can be at least one of ambient air and nitrogen. The enclosure can be partially open on one or more sides of the enclosure in some implementations. As another example, the quality characteristic can include a ripeness stage, stem rot, desiccation, taste, internal rot, mold, and firmness. The one or more food items can include avocados, and the volatiles outgassed by the avocados can include one or more of: aldehydes, alcohol, and terpenes. As yet another example, the one or more food items can include mandarins, and the volatiles outgassed by the mandarins can include one or more of: ethanol, ethyl acetate, ethyl-esters, acetaldehyde, alpha-pinene, limonene, linalool, germacrene D, and beta-farnesene.

As another example, the historic supply chain information can include at least one of a place of origin of the other food items, a storage temperature of the other food items, shipping conditions associated with the other food items, and historic ripening information associated with the other food items. In some implementations, determining the quality characteristic of the one or more food items can include mapping the volatiles in the output data to one or more quality features that are identified from (i)-(iii).

In some implementations, the computing system can also identify supply chain information for the one or more food items that can include a preexisting supply chain schedule and destination for the one or more food items, determine whether to modify the supply chain information for the one or more food item based on the determined quality characteristic of the one or more food items, and in response to a determination to modify the supply chain information, generate modified supply chain information based on the determined quality characteristic. The modified supply chain information can include one or more of a modified supply chain schedule and modified destination for the one or more food items.

One or more embodiments can also include a method for generating a trained model to determine a quality characteristic of a food item. The method can include receiving, by a computing system, (i) volatiles data of a first plurality of food items that have a quality characteristic, (ii) volatiles data of a second plurality of food items that do not have the quality characteristic, (iii) human observations of other food items, and (iv) historic supply chain information associated with the other food items. The first plurality of food items, the second plurality of food items, and the other food items can be a same food type. The method can also include generating, by the computing system, a volatiles marker profile based on performing random forest modeling on (i) and (ii). The volatiles marker profile can indicate one or more volatiles whose concentrations can be present in the first plurality of food items but not in the second plurality of food items. The method can also include generating, by the computing system, a machine learning model based on mapping the volatiles marker profile to quality characteristics identified by (iii) and (iv). The machine learning model can correlate presence and concentrations of the volatiles with one or more quality characteristics of food items of the same food type.

In some implementations, the volatiles data can be non-destructively captured from the first and second plurality of food items.

One or more embodiments can also include a method for producing a volatile ripening marker timeline associated with agricultural products. The method can include separating, by a computing system, a plurality of an agricultural product into n groups. Each group of the n groups can represent a different ripeness stage of the agricultural product and n can be an integer greater than 1. The method can also include independently assessing, by the computing system, presence and concentrations of one or more volatile compounds that are outgassed from each group of the n groups, and producing, by the computing system, a volatile marker profile for the agricultural product. The volatile marker profile can indicate the one or more volatile compounds whose presence and concentrations are identified in some but not all of the n groups. The method can also include generating, by the computing system, a volatile ripening marker timeline. Each ripening stage represented in the volatile ripening marker timeline can be correlated with the one or more volatile compounds in the volatile marker profile.

The one or more embodiments described herein can include one or more of the following features in some implementations. For example, the method can also include assessing, by the computing system, presence and concentrations of volatile compounds outgassed from an agricultural product based on comparing the presence and concentrations of the volatile compounds outgassed from the agricultural product to the volatile marker profile in the volatile ripening marker timeline. As another example, the method can include predicting, by the computing system, a current ripeness stage of the agricultural product based on the volatile ripening marker timeline.

In some implementations, the method can also include controlling, by the computing system, a ripening process of the agricultural product based on the predicted current ripeness stage of the agricultural product. As another example, the volatile ripening marker timeline can include a plurality of volatile marker profiles associated with each of a plurality of n stages of ripeness of the agricultural product. Each of the plurality of volatile marker profiles can represent one or more volatile compounds whose presence and concentrations indicate a stage of ripeness as compared to other stages of ripeness. N can be an integer greater than 1.

The devices, system, and techniques described herein may provide one or more of the following advantages. For example, supply chain modifications can be made before agricultural products are delivered to consumers, which can reduce product-based waste. Identifying quality of products as early as possible in the supply chain lifecycle can be advantageous to appropriately move the products down the supply chain with minimal or no product-based waste. The disclosed technology can be used to determine whether an agricultural product is of a suitable quality to be sold or otherwise delivered to consumers. The disclosed technology can also be used to predict when the product's quality will deteriorate (e.g., when the product will mold). If the product is not of suitable quality or the product is predicted to be of lower quality within a threshold time period, then supply chain modifications can be made in relation to that product. For example, the product can be delivered to grocery stores faster than other products such that the product can be consumed before the product's quality lowers. The product can also be sold at a lower price since it is of lower quality. Similarly, the disclosed technology can be used to segment high quality products from low quality products. The high quality products can be sold at higher prices than the low quality products. As another example, the product can be delivered to a food processing plant to be used in production of processed foods. As a result, the low quality product can still be used instead of becoming waste.

Therefore, the disclosed technology can benefit numerous stakeholders in the supply chain lifecycle. The disclosed technology can benefit producers by maintaining harvest quality, providing inventory management control, and offering brand differentiation. Distributors can benefit from new markets, new modes of transportation, and quality arrival of products. Retailers can benefit from less product shrinking, fresher shelf appearance, higher shopper satisfaction, and increased shopper loyalty and repeat visits. Moreover, consumers (e.g., shoppers) can benefit from fresher products, more time to enjoy the products, less spoilage, and less money wasted on products that spoil faster.

As another example, the disclosed technology can be portable, which can make it easier and faster to determine product quality. For example, an apparatus for testing product quality can include a tube having an sorbent material. The tube can be easily and quickly inserted into chambers that house agricultural products. The sorbent material of the tube can collect volatiles associated with the products and then the tub can be inserted into a gas chromatograph machine for immediate processing. Thus, the tube having the sorbent material can be moved around a distribution center and used to capture volatiles associated with products that are stored in different rooms, containers, or locations in the distribution center.

As another example, the disclosed technology can provide for measuring product quality in batches, which can make it easier and faster to determine supply chain modifications for batches of products. As described herein, multiple sniffers (e.g., tubes) having the sorbent material can be placed and positioned throughout the distribution center (e.g., along conveyor belts, in rooms, containers, or other storage locations). The multiple sniffers can capture volatiles from products that are located near the sniffers. The volatiles can then be analyzed to determine an aggregate quality of the products in batch. The aggregate quality can be used to determine how to move the batch of products as a unit in the supply chain.

As yet another example, the disclosed technology can provide for modeling different quality characteristics of products such that product quality can be predicted more accurately and earlier in the supply chain life cycle. Different classifiers can be generated and used in training the disclosed technology to predict a plurality of different quality characteristics of products. The more quality characteristics that are identified, the more likely that value of the products can be maximized and product-based waste can be mitigated or avoided. For example, using modeling and machine learning training techniques, the disclosed technology can provide for predicting ripeness, stem rot (and different dimensions or stages of rot), flavor (e.g., sweet or sour), and vascular browning before the products leave the distribution center. Based on these predictions, modifications can be made to the supply chain such that the products' value can be maximized.

The details of one or more implementations are set forth in the accompanying drawings and the description below. Other features and advantages will be apparent from the description and drawings, and from the claims.

### BRIEF DESCRIPTION OF THE DRAWINGS

FIG. 1A is a conceptual diagram for determining agricultural product quality using techniques disclosed herein.

FIG. 1B is a block diagram of a system for determining a ripeness stage of an agricultural product.

FIG. 1C is a flowchart of a process for determining a ripeness stage of a group of agricultural products.

FIG. 2 is an example apparatus for collecting volatiles of agricultural products to determine quality of the agricultural products.

FIG. 3 is an example system for collecting volatiles of agricultural products to determine quality of the agricultural products.

FIG. 4 is a conceptual diagram for generating a trained model that can be used for determining agricultural product quality.

FIG. 5 is a flowchart of a process for determining agricultural product quality.

FIG. 6 is a flowchart of a process for generating a trained model that can be used for determining agricultural product quality.

FIG. 7 is a flowchart of a process for using the trained model to determine agricultural product quality in real-time.

FIG. 8 is a flowchart of a process for determining supply chain modifications based on determined agricultural product quality.

FIG. 9 is a schematic diagram of an exemplary GC×GC set up.

FIG. 10 depicts an exemplary sample collection apparatus for collecting volatiles of agricultural products.

FIG. 11 is a graphical depiction summarizing an exemplary volatile sample collection comparison of thermal desorption (TD), solid-phase microextraction (SPME), and hisorb.

FIG. 12 depicts an exemplary Scores plot and Biplot of two principle components to demonstrate how measured components relate to agricultural products.

FIG. 13 depicts Partial Least Squares-Discriminant Analysis (PLS-DA) results using groups labeled control and inoculated agricultural products.

FIG. 14 depicts a representative chromatogram of test agricultural product.

FIG. 15 is a graphical depiction representing average concentration of group types over a lifetime of an example avocado.

FIG. 16A is a flowchart of a process for analyzing low abundance volatiles that can be outgassed by example mandarins.

FIG. 16B shows graphical depictions of identified volatiles that are outgassed by the example mandarins in FIG. 16A.

FIGS. 17A-B is a flowchart of a process for identifying volatile compounds that can be related to off-flavor development in agricultural products.

FIG. 18 is a schematic diagram that shows an example of a computing device and a mobile computing device.

Like reference symbols in the various drawings indicate like elements.

### DETAILED DESCRIPTION OF ILLUSTRATIVE EMBODIMENTS

This document generally describes systems, methods, and techniques to non-destructively assess quality and/or flavor characteristics of agricultural products before distributing them to consumers or other stakeholders in a supply chain. For example, the disclosed technology can provide for producing a volatile marker profile associated with a specific characteristic and/or trait of an agricultural product. The characteristic and/or trait can include presence of an infection, flavor characteristics, and/or ripening stage of the agricultural product.

In some implementations, volatile marker profiles can be produced by comparing presence and/or amount of volatile compounds outgassing from a plurality of the agricultural product having a specific trait and/or characteristic with the presence and/or amount of volatile compounds outgassing from a plurality of the same agricultural product lacking that trait and/or characteristic. The volatile marker profile can represent one or more of the volatile compounds whose presence and/or amount characterizes the plurality of agricultural products having a specific trait and/or characteristic, but not the plurality of agricultural products lacking the specific trait and/or characteristic.

In some implementations, the disclosure is directed to a volatile marker profile associated with infection in the agricultural product, the profile representing one or more of the volatile compounds whose presence and/or amount characterizes presence of the infection in the agricultural product. The disclosure can also provide for segregating agricultural products that are identified as infected from uninfected agricultural products. Supply chain modifications can then be appropriately determined for the infected and uninfected agricultural products before such products are delivered to end-consumers or other relevant stakeholders. Segregating infected agricultural products from uninfected agricultural products can be beneficial to reduce a spread of the infection and potential agricultural product-based waste. Segregating the products can also allow for treatment of the infected products to avoid or mitigate waste. Treatment can be, for example, an anti-microbial treatment, withholding ethylene treatment, prescribing ethylene treatment, and/or one or more other forms of proactive or reactive treatment processes.

In some implementations, the plurality of volatile marker profiles can be used to construct a timeline associated with development (e.g., ripening stages) of the agricultural product. The timeline can include the profiles associated with each of n stages in a ripening process. The profiles can represent one or more of the volatile compounds whose presence and/or amount characterizes stages of ripeness as compared to other stages of ripeness. Moreover, n can be an integer greater than 1. The timeline can indicate how much time may be needed for the particular agricultural product to ripen.

As an example, an amount of terpenes and alcohols produced by an agricultural product can decrease over time. Terpenes and alcohols can be detected in an amount of approximately 700 ppm w/w outgassed from unripe avocados. That amount can decrease to below approximately 100 ppm w/w in a ripe avocado. Thus, a volatile marker profile of an unripe avocado can have a higher concentration of terpenes and/or alcohols than a volatile marker profile of a ripe avocado. Therefore, comparing the volatile compound profile of an avocado of unknown ripeness with the volatile marker profiles of avocados with known ripeness can be used to determine ripening progress of the avocado of unknown ripeness.

The predicted ripening timeline of the agricultural product can also be used to further control a ripening rate of the agricultural product. In some implementations, the ripening rate can be controlled by altering storage conditions and/or prescribing ethylene or other treatments to the agricultural product. For example, ripening progress can be slowed by storing the agricultural product in refrigerated conditions. As another example, ripening progress can be accelerated by treating the agricultural product with, for example, ethylene. As an additional example, ripening progress can be slowed by treating the product with an ethylene inhibitor, blocker or absorber.

In some implementations, the volatile marker profiles can be used to provide information about taste that an unripe agricultural product will have once the product ripens. Ripe flavor marker profiles can represent one or more volatile compounds whose presence and/or amount characterizes a ripe flavor characteristic of the agricultural product. The flavor characteristics that can be assessed can include one or more of how sweet, bitter, tart, juicy, alcoholic, pungent, green, grassy, fruity, citrusy, leafy, terpy, and/or woody the agricultural product is.

As an example, presence and/or amount of aldehydes, which are generally associated with positive flavor attributes, produced by an agricultural product can remain relatively constant through a duration of a ripening process. Accordingly, detection and quantification of the aldehydes produced by an unripe avocado, for example, can allow for a determination of the flavor characteristics of the avocado when it ripens. Thus, as described herein, presence and/or amount of certain types of compounds can influence the flavor and/or odor characteristics of the agricultural product. Alcohols can be responsible for alcoholic, pungent, and/or green flavors; aldehydes can be responsible for green, fruity, and/or citrusy flavors; unsaturated aldehydes can be responsible for green, fruity, and/or leafy flavors; and terpenes can be responsible for citrusy, terpy, and woody flavors.

Thus, using the plurality of volatile marker profiles, the disclosed technology provides for determining characteristics of the agricultural product early in the supply chain. Based on the determined characteristics, changes can be made to the supply chain such that the agricultural product can be consumed or otherwise used with minimal to no agricultural product-based waste.

As described herein, production of volatile marker profiles can be non-destructive. In other words, agricultural product characteristics can be determined using non-destructive volatile collection techniques. As a result, the agricultural products may not be compromised, cut, sliced, chopped, mashed, peeled, blended, or otherwise destroyed. The agricultural products can remain unaltered when delivered to end consumers or other relevant stakeholders in the supply chain.

Presence and/or amount of the volatile compounds outgassed by an agricultural product can be detected in a variety of ways. For example, and as described throughout this disclosure, the agricultural product can be inserted into a closed container for a period of time (e.g., 30 minutes or more) to allow the container to fill with the volatile compounds produced by the agricultural product (e.g., terpenes, saturated and unsaturated aldehydes, alcohols and hydrocarbons). Methods such as solid-phase microextraction (SPME) and/or thermal (TD) desorption can be used to extract the different volatile compounds (e.g., analytes) associated with the flavor, quality, and/or ripeness of the agricultural product from the container. Desorption of analytes, for example, into a chromatographic technique (e.g., two-dimensional gas chromatography (GCxGC)) can be implemented to separate the analytes to produce a chromatogram. From the chromatogram, group-type quantification of the volatile compounds, such as terpenes, saturated and unsaturated aldehydes, alcohols and hydrocarbons can be made. One or more trained machine learning models can also be used to quantify and classify different volatile compounds.

In some implementations, production of a volatile marker profile for a particular trait and/or characteristic of an agricultural product can include comparing the volatile compound profiles of a plurality of agricultural products. The produced volatile marker profile can further be tuned or enhanced as more volatile compound profiles of the agricultural product with the same trait and/or characteristic are analyzed. Machine learning or other similar methods and techniques can be used to update and refine volatile marker profiles for particular traits and/or characteristics of the agricultural product. Using the refined volatile marker profiles, traits and/or characteristics of the agricultural product can be more accurately determined earlier in the supply chain lifecycle. As a result, modifications to the supply chain can be made earlier on to optimize on the traits and/or characteristics of the agricultural product and to avoid or otherwise mitigate agricultural product-based waste.

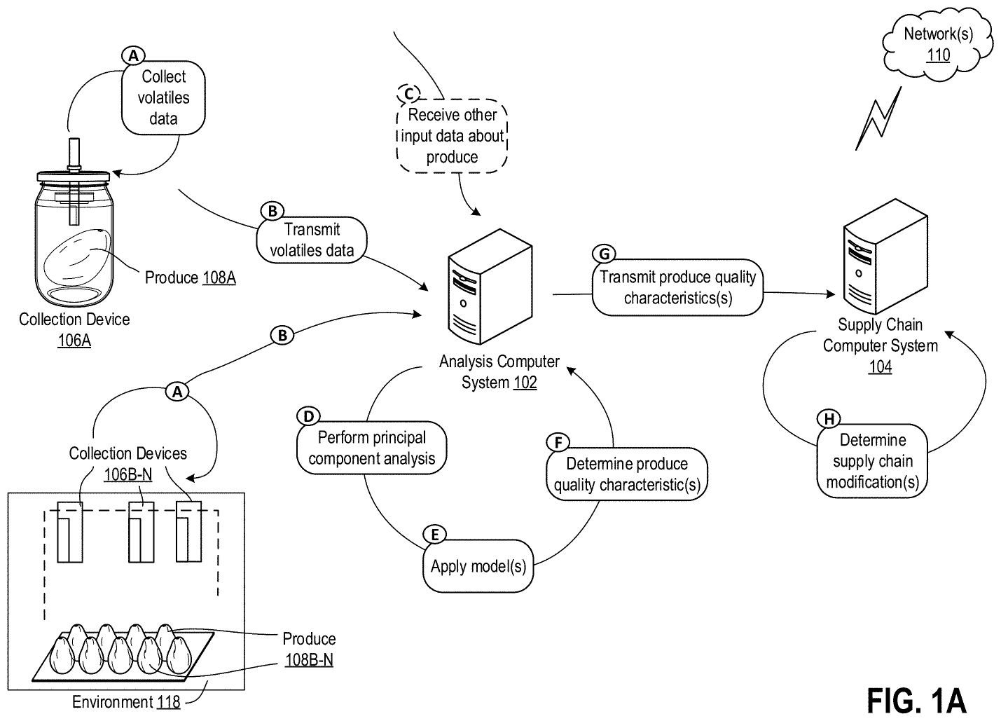

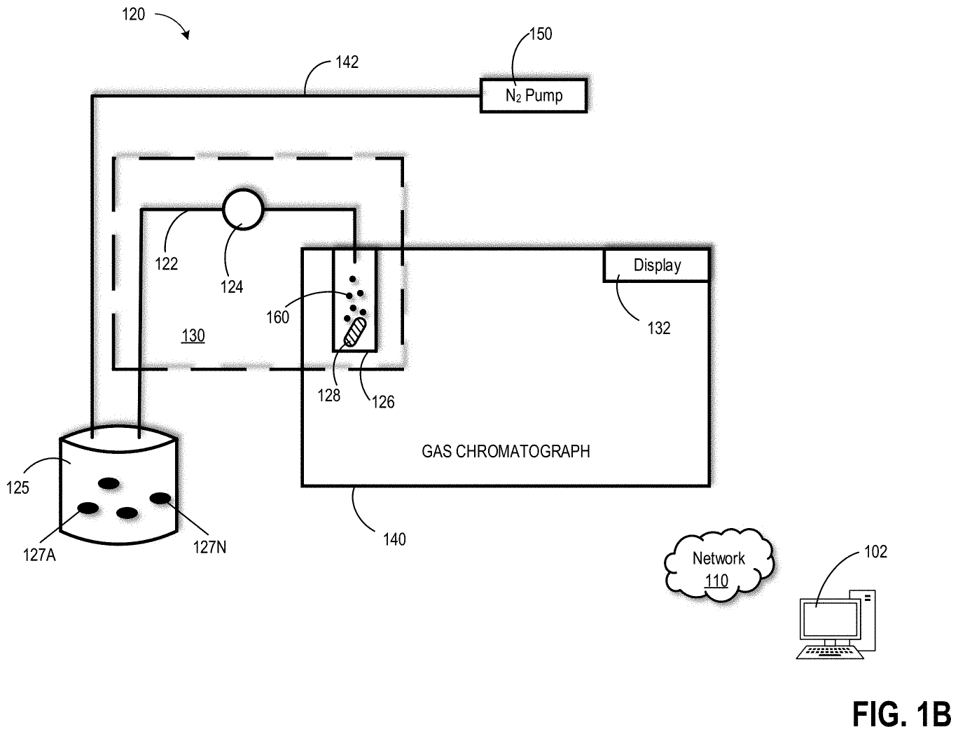

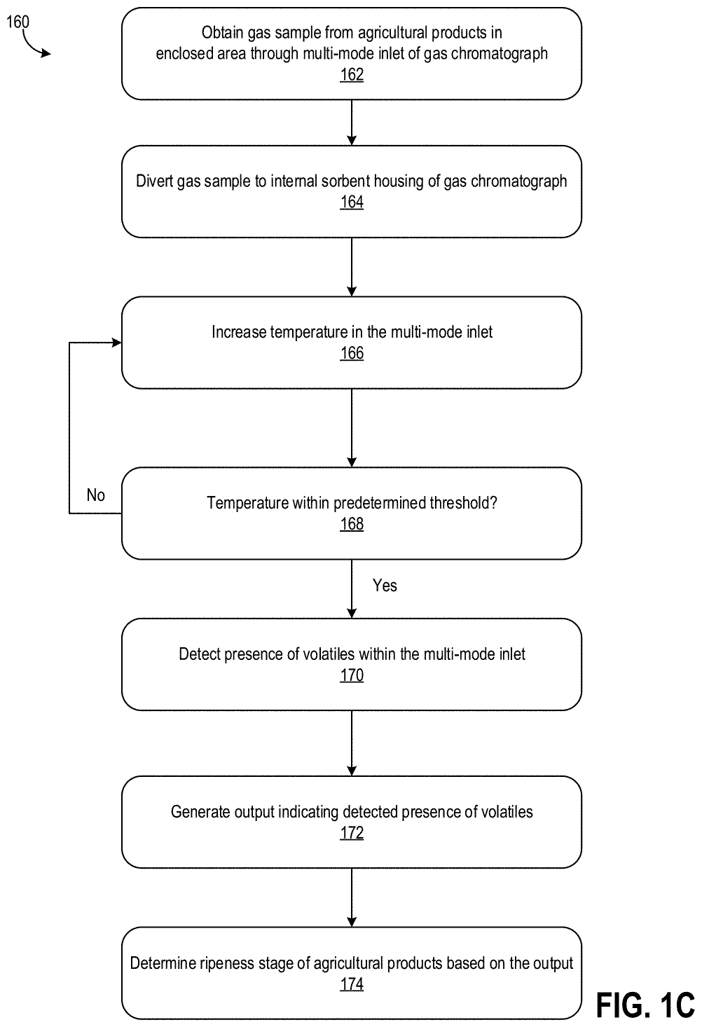

Referring to the figures, FIG. 1A is a conceptual diagram for determining agricultural product quality using techniques disclosed herein. Agricultural products can be tested for quality characteristics without being destroyed, altered, or changed. The quality characteristics can include ripeness levels of stages, prediction of stem rot, propensity for mold growth, flavor (e.g., sweet, sour, tart, etc.), internal qualities such as vascular browning, smell, and nutritional content. The quality characteristics can be predicted and determined early in a supply chain lifecycle such that supply chain decisions can be made to optimize agricultural product qualities and reduce or mitigate product-based waste.

For example, if an agricultural product is predicted to be at a preferred ripeness stage, then supply chain decisions can be made early on that direct that product to be delivered to grocery stores that are geographically closest to a current location (e.g., storage facility, farm) of the product. As a result, the product can be given to end-consumers earlier, while the product is at the preferred ripeness stage. The product can also be sold at a higher price. On the other hand, if the product is predicted to develop stem rot or vascular browning within a certain timeframe, supply chain modifications can be made early on in the supply chain lifecycle to redirect the product to a food processing plant. After all, the product may not be suitable for consumption by a time that it arrives to end-consumers and/or the product may be sold at a lower price or otherwise not purchased by end-consumers. Thus, a preferred supply chain modification for the product can be to direct the product to the food processing plant, where its projected quality characteristics may not cause product-based waste.

As described herein, agricultural products can be tested by capturing volatiles that are outgassed by such products. The volatiles can be analyzed and mapped to different quality features using machine learning techniques. A disappearance, appearance, or accumulation of volatiles can provide valuable information about quality of such agricultural products.

Still referring to FIG. 1A, an analysis computer system 102 can be in communication (e.g., wired, wireless) with a supply chain computer system 104 and collection devices 106A-N via network(s) 110. The analysis computer system 102 and the supply chain computer system 104 can be separate systems. In some implementations, the analysis computer system 102 and the supply chain computer system 104 can be a same computing system. Moreover, the analysis computer system 102 can include a gas chromatograph machine for processing the volatiles. In some implementations, the analysis computer system 102 can be in communication (e.g., wired, wireless) with a gas chromatograph machine.

The collection devices 106A-N can include sorbent material that allows for the capturing of volatiles that are outgassed by produce 108A-N (e.g., agricultural products). In this example, the produce 108A-N, or agricultural products, are avocados. The produce 108A-N can include any other product, including fruits such as berries, apples, and limes, as well as vegetables, produce, and meats.

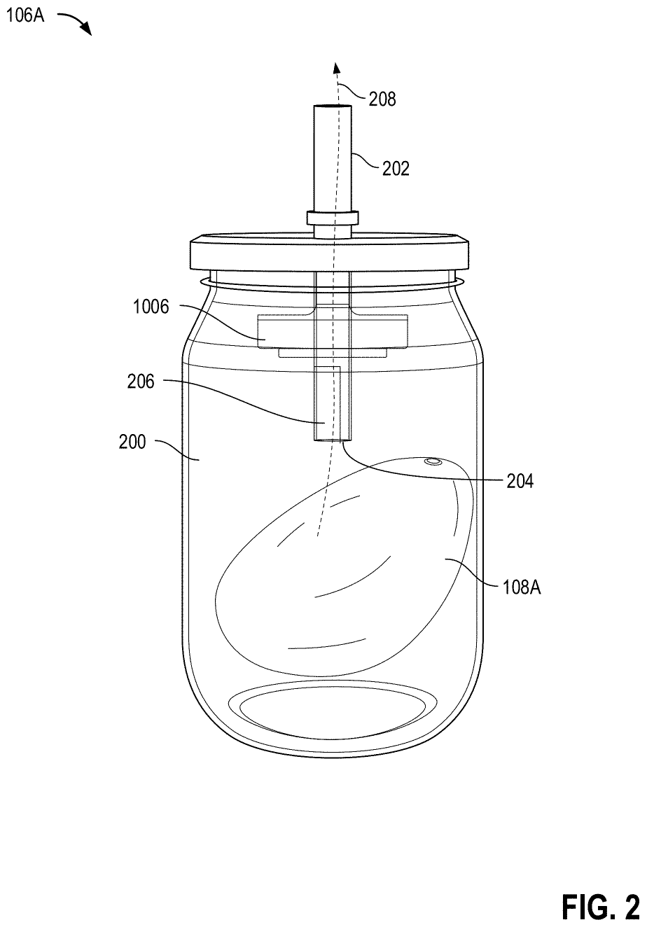

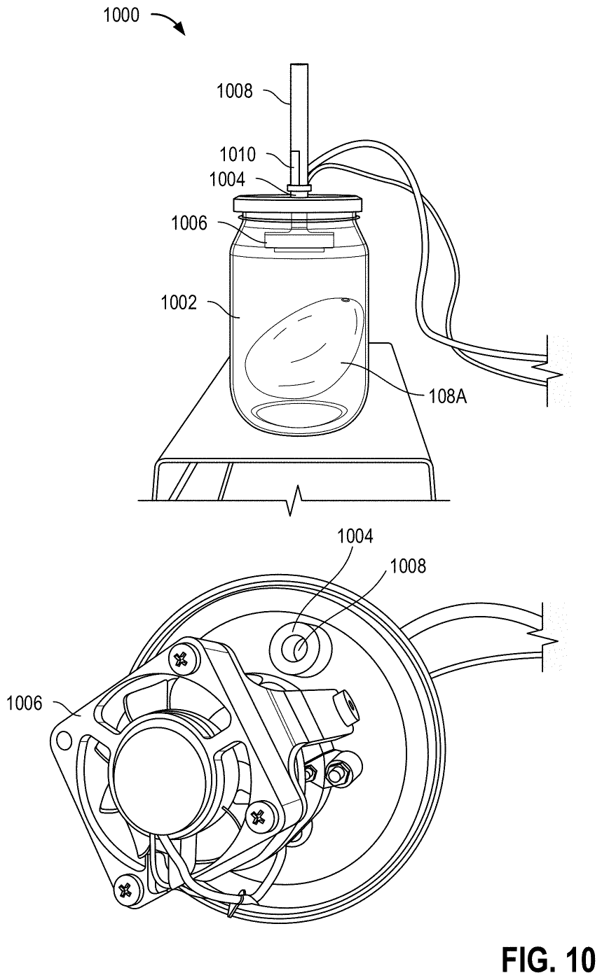

In some implementations, the collection device 106A can be placed in a container or similar controlled atmosphere storage are. The container can contain one produce 108A. In some implementations, the container can house multiple produce of a same type. The collection device 106A can be configured to collect volatiles that are outgassed from the produce 108A (step A) (e.g., refer to FIGS. 2, 10). In some implementations, air can flow into the collection device 106A and/or the container to promote capturing of the volatiles, as described further below.

In some implementations, multiple collection devices 106B-N can be placed in an environment 118. These collection devices 106B-N can be configured to collect volatiles that are outgassed from one or more batches of produce 108B-N (step A). Therefore, aggregate quality characteristics can be determined for the produce 108B-N. Sometimes, individual quality characteristics can also be determined for each of the produce in the produce 108B-N. The environment 118 can be a storage location, a room in a storage facility (e.g., a cold storage area), a shipping container, or another similar location. The produce 108B-N can be stored on a pallet, in a bin, and/or container.

Once volatiles are collected by the collection devices 106A-N within a collection timeframe, the collected volatiles can be transmitted to the analysis computer system 102 (step B). For example, the collection devices 106A-N, having the volatiles attached to the sorbent material, can be placed within a gas chromatograph that is in communication with or part of the analysis computer system 102.

The analysis computer system 102 can be configured to analyze collected volatiles and determine/predict quality characteristics of agricultural products. The analysis computer system 102 can include the gas chromatograph machine or similar components. The analysis computer system 102 can perform desorption techniques to remove the volatiles from the sorbent material (e.g., by applying thermal energy to the sorbent material) and/or to otherwise analyze the collected volatiles. Desorption techniques can include thermally desorbing the volatiles after increasing a temperature within the collection device 106A-N. The analysis computer system 102 can thus perform principal component analysis by separating the volatiles into components and/or groups, based on the volatiles' presence and concentrations (step D). Using gas chromatography techniques, the analysis computer system 102 can detect abundances of each of the volatiles and/or groups of volatiles. Such information can be used, by the analysis computer system 102, to correlate each of the volatiles and/or groups of volatiles with quality characteristics of the agricultural products.

The analysis computer system 102 can also apply one or more machine learning trained models to the volatiles data (step E). The models can be trained to classify and correlate volatiles with different quality characteristics associated with the produce 108A-N. Models can be generated to determine and predict different quality characteristics associated with the produce 108A-N. Moreover, different models can be generated for different types of produce. Using the models, the analysis computer system 102 can determine and predict quality characteristics of the produce 108A-N (step F).

In some implementations, the analysis computer system 102 can also determine the quality characteristics of the produce 108A-N using additional input data about the produce 108A-N (step C). The analysis computer system 102 can receive the additional input data from one or more relevant stakeholders in the supply chain. The additional input data can include a place of origin, a storage temperature, a transit temperature, other storage conditions, other transit conditions, and/or historical information about the produce 108A-N (e.g., previous ripening cycles/analysis, previous volatiles data for produce of a same type as the produce 108A-N). Using the additional input data with the models can be advantageous to develop more robust and accurate non-invasive determinations and predictions of produce quality characteristics (step F).

Once determined and predicted, the analysis computer system 102 can transmit the quality characteristics of the produce 108A-N to the supply chain computer system 104 (step G). The supply chain computer system 104 can be configured to determine supply chain modifications based on the quality characteristics of the produce 108A-N (step H). The modifications can be automatically determined by the supply chain computer system 104. In some implementations, relevant stakeholders in the supply chain can view the produce quality characteristics (e.g., at a display of a user mobile device, such as a computer, laptop, tablet, cellphone, etc.) and provide user input indicative of one or more modifications to the supply chain. In some implementations, the supply chain computer system 104 can determine supply chain modifications and present them to the relevant stakeholders for review. The stakeholders can then approve, reject, and/or edit the supply chain modifications.

Steps A-H can be performed at any point in the supply chain lifecycle. Volatiles can be collected early in the lifecycle in order to better understand quality characteristics of the produce as the produce ripens. Steps A-H can be performed throughout a ripening lifecycle of the produce. Steps A-H can also be performed multiple times during the ripening and/or supply chain lifecycles. For example, steps A-H can be performed at a first time once the produce 108A-N arrives at a storage facility. During this first time, a ripening lifecycle of the produce 108A-N can be predicted. Then, steps A-H can be performed second, third, fourth, etc. times at different stages of the predicted lifecycle of the produce 108A-N. The steps A-H can also be performed a last time before the produce 108A-N is moved out of the storage facility to end-consumers. Performing the steps A-H multiple times throughout the supply chain and/or ripeness lifecycles can be advantageous to (1) collect an abundance of data about the produce 108A-N that can be used to enhance trained machine learning models, volatile analysis, and quality characteristics predictions and determinations, (2) make supply chain modifications early in the lifecycles, (3) optimize the produce 108A-N's quality characteristics, and (4) avoid or mitigate produce-based waste.

FIG. 1B is a block diagram of a system 120 for determining a ripeness stage of an agricultural product. The system 120 can include an enclosure 125, a multi-mode inlet 130, a gas chromatograph 140, gas pump 150, and the analysis computer system 102. In some implementations, one or more aspects of the depicted system 120 can be integrated into a single device or computing system, such as the gas chromatograph 140. The network 110 can provide for communication (e.g., wired and/or wireless) between one or more components described herein.

The enclosure 125 can be any type of enclosure, container, and/or storage room as described throughout this disclosure. The enclosure 125 can be used to store agricultural products 127A-N and to collect volatiles released by such products. In some implementations, for example, the enclosure 125 can include a single container of one or more agricultural products. In some implementations, an enclosure 125 can include any enclosed area such as a shipping container, a cargo hold of a transportation vehicle, or a ripening room.

The multi-mode inlet 130 can be an apparatus that can capture a gas sample from the enclosure 125 and determine whether a level of volatiles 160, if any, are included in the captured gas sample. The multi-mode inlet 130 can include a sampling line 122, one or move valves 124, a sorbent material housing 126, and a sorbent material 128. At least the sorbent material housing 126 that houses the sorbent material 128 can be installed within the gas chromatograph 140. The sampling line 122 can extend from the multi-mode inlet 130 into the enclosure 125. In some implementations, the multi-mode inlet 130 can deploy the sampling line 122 as a retractable appendage into the enclosure 125 for a predetermined amount of time. After the predetermined amount of time, the sampling line 122 can be retracted from the enclosure 125 and then, subsequently, be deployed into another enclosure. In some implementations, the sampling line 122 can be a tube, pipe, or other appendix that is fixed in place a particular enclosure 125 and used to continually capture gas samples (e.g., volatiles 160) from the enclosure 125 over time.

The multi-mode inlet 130 can obtain a gas sample from the enclosure 125. In some implementations, the multi-mode inlet 130 can obtain the gas sample from the enclosure 125 via manipulation of one or more valves 124. For example, the multi-mode inlet 130, or other component of the system 120, can cause the one or more valves 124 to open, thus allowing gas from the enclosure 125 to flow into the sorbent material housing 126 via the one or more open valves 124. This gas from the enclosure 125 can include volatiles 160 that have been emitted from the one or more agricultural products 127A-N into the gases of the enclosure 125 during natural ripening of the products 127A-N. The system 120 can also be configured to assist gas flow from the enclosure 125 into the sorbent material housing 126. For example, the system 120 can include the pump 150 to pump another gas into the enclosure 125 via a gas line 142. The gas pumped into the enclosure 125 by the pump 150 can displace gas inside the enclosure 125 into the sorbent material housing 126 via the sampling line 122. In some implementations, the gas pumped into enclosure 125 can include Nitrogen (N₂) at flow rates ranging from 15 mL/min to 50 mL/min.

The sorbent material 128 inside the sorbent material housing 126 can be of a composition that is designed to absorb volatiles that have been carried into the sorbent material housing 126 by the displaced gas. In some implementations, the sorbent material can include 0.07 g to 0.14 g of TENAX. Other substances can also be used as the sorbent material. Such substances can be selected based on their characteristics that can relate to absorption of volatiles, reactions to applied heat, desorption properties, etc. In some implementations, changing the amount of the material, such as 0.07 g of TENAX instead of 0.14 g of TENAX, can optimize the system 120 for improved performance. For example, volatiles can be absorbed within 60 minutes when nitrogen is pumped in at a flow rate of 50 mL/min.

The gas chromatograph 140 can include a heating element that can be used to apply heat to the sorbent material 128, the sorbent material housing 126, or a combination of both. The applied heat can be used to cause the sorbent material 128 to desorb the volatiles 160 that were previously absorbed into the sorbet material 128 during sampling. In some implementations, the gas chromatograph 140 can be configured to heat the sorbent material 128, the sorbent material housing 126, or both, above a predetermined threshold temperature. This predetermined threshold temperature can be selected based on properties of the sorbent material, the volatiles to be detected, the food type, or a combination thereof.

The gas chromatograph 140 can be configured to detect the presence or absence of volatiles 160 desorbed, or otherwise expelled, from the sorbent material 128. The gas chromatograph 140 can also determine a level of each detected volatile that was present in the sorbent material housing 126. The level of each detected volatile can include a number of a type of volatiles detected, a concentration of volatiles detected, or the like. The gas chromatograph 140 can be configured to generate output data that indicates a level of each detected volatile within the sorbent material housing 126.

The analysis computer system 102 can process instructions stored in a memory of the gas chromatograph 140 that, when executed, cause one or more processors to analyze the detected volatiles. For example, the system 102 can determine an overall ripeness stage for the agricultural products 127A-N. In some implementations, the level of each detected volatile can be mapped to a predetermined ripeness stage. In some implementations, the system 102 can associate each ripeness stage with a predetermined volatile level range. Mapping the level of each detected volatile to a ripening stage can be achieved by comparing, for each detected volatile, the level of the detected volatile to the volatile range for each ripening stage. A level of a detected volatile can satisfy a volatile range during this comparison if the level of the detected volatile falls within the volatile range.

FIG. 1C is a flowchart of a process 160 for determining a ripeness stage of a group of agricultural products. The process 160 can be performed by one or more computing systems, such as the analysis computer system 102. Moreover, one or more blocks in the process 160 can be performed by the gas chromatograph 140 (e.g., refer to FIG. 1B). As described herein, in some implementations, the analysis computer system 102 and the gas chromatograph 140 can be one computer system, server, or network of computers, servers, and/or devices. For illustrative purposes, the process 160 is described from the perspective of the analysis computer system 102.

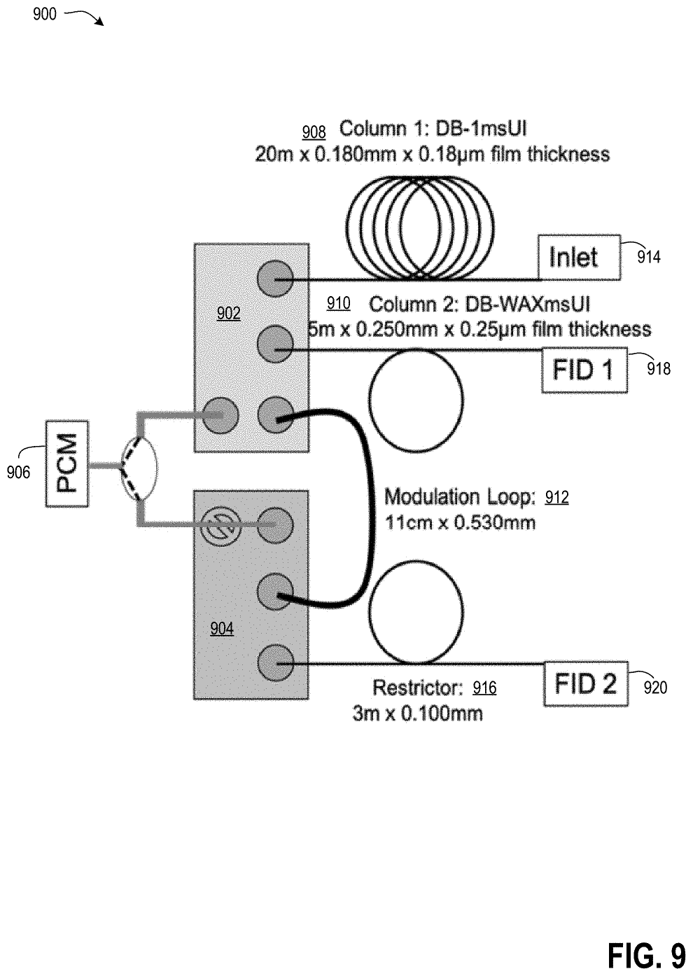

Referring to the process 160 in FIG. 1C, the analysis computer system 102 can obtain a gas sample from agricultural products in an enclosed area in 162. The analysis computer system 102 can use a multi-mode inlet coupled to the gas chromatograph to obtain the gas sample (e.g., refer to FIGS. 1B, 9). In some implementations, the multi-mode inlet can obtain the gas sample by opening a valve that is coupled to pathway that extends towards a sorbent material housing (e.g., refer to FIG. 1B). To obtain the gas sample, in some implementations, the analysis computer system 102 can instruct a gas pump to inject a gas, such as nitrogen, into the enclosed area to capture volatiles that are outgassed by the agricultural products and displaced by the injected gas.

As described throughout this disclosure, the enclosed area can include a single container housing one or more agricultural products (e.g., refer to FIGS. 1A, 2, 3). An appendage can be coupled to the multi-mode inlet and extended into the single container. The appendage can include a valve that can be configured to enable flow of gas into a pathway that extends towards the sorbent material housing of the gas chromatograph. Thus, volatiles that are outgassed from the one or more agricultural products can travel through the appendage and collect on the sorbent material housing of the gas chromatograph. The enclosed area can also be a variety of other spaces, including but not limited to a ripening room, a shipping container, a storage unit, or any similar area for housing one or more agricultural products.

In some implementations, the multi-mode inlet can be configured to sample gas for a predetermined amount of time (e.g., predetermined sampling time). Sometimes, the predetermined amount of time can be the same regardless of a type of the agricultural products. As described herein, the predetermined amount of time can be based on a material and/or composition of the sorbent material housing.

The analysis computer system 102 can divert the gas sample to an internal sorbent housing of the gas chromatograph in 164. The gas sample can be diverting using one or more gas sampling valves of the gas chromatograph. The internal sorbent housing can include a sorbent material such as TENAX. In some implementations, one gas sampling valve can be employed. That is, the multi-mode inlet can have (i) an outer opening that does not have a valve and (ii) an inner valve that seals off the internal sorbent material housing. In such implementations, the gas sampling valve can be opened once the multi-mode inlet obtains the gas sample. The gas sampling valve can then be closed after a predetermined amount of time has passed, thereby allowing the gas to enter the internal sorbent material housing. In some implementations, the internal sorbent material housing can be made of glass and/or can be a tube-like structure.

In some implementations, the multi-mode inlet can have multiple gas sampling valves. For example, the analysis computer system 102 can instruct the multi-mode inlet to close an internal valve and open an external valve, thereby allowing gas to enter an initial chamber of the multi-mode inlet. Then, the external value can be automatically closed prior to opening the internal value to retain the captured gas in the initial chamber of the multi-mode inlet. After the sampled gas has been captured in the initial chamber, the internal valve can be automatically opened with the external valve closed to allow the sampled gas to be diverted into the internal sorbent material housing. The internal valve, which can be a valve of a single valve or dual-valve system, can be automatically closed to contain the sampled gas within the internal sorbent material housing. Once the internal sorbent material housing has been closed off using the one or more values, volatiles captured in the gas of the internal sorbent material housing can be absorbed into the sorbent material.

The analysis computer system 102 can increase temperature in the multi-mode inlet of the gas chromatograph in 166. Temperature can be increased using a heating element that is installed within the sorbent material housing or a heating element that is installed on the exterior of the sorbent material housing. In some implementations, the heating element can directly contact the sorbent material housing. The heating element can also heat a substance such as water that envelops the sorbent material housing. The heated substance can then cause an increase in temperature of a sorbent, the area within the sorbent material housing, or a combination thereof.

Heating of the internal sorbent housing can cause the temperature of the sorbent material to increase. Increasing the temperature of the sorbent material can be beneficial to desorb the captured volatiles such that they are released from the sorbent material and can be processed. The sorbent material can have properties that cause volatiles absorbed into the sorbent material to be desorbed, or otherwise expelled, into an open portion of the internal sorbent housing after the sorbent material is heated beyond a threshold level.

Next, the analysis computer system 102 can determine whether the temperature in the inlet is within a predetermined threshold (168). The temperature threshold can be determined based on the sorbent material, the type of food item being analyzed, the type of volatile, or any combination thereof. Different sorbent materials may be associated with different temperature thresholds that, when satisfied, cause the sorbent material to desorb, or otherwise, expel, into the open portion of the internal sorbent material housing. If the temperature is not within the predetermined threshold, then the analysis computer system 102 can continue to increase the temperature in the multi-mode inlet in 166. If the temperature is within the predetermined threshold, then the analysis computer system 102 can detect presence of volatiles within the multi-mode inlet in 170, as described herein.

Moreover, the analysis computer system 102 can generate output indicating the detected presence of volatiles (172). The output can include data that identifies each volatile that is detected. The output can also include data that identifies absence of one or more volatiles that were expected to be present in the gas sample. The output data can also provide an indication of levels of each volatile that was detected.

The analysis computer system 102 can also determine a ripeness stage of the agricultural products based on the output in 174. As described further throughout this disclosure, the ripeness stage can be determined based on levels or concentrations of each detected volatile. Moreover, the detected volatiles can be grouped together into classes and analyzed to determine how the classes of volatiles correlate to different ripeness stages. Using machine learning trained models, the levels or concentrations of detected volatiles can be mapped to predetermined ripeness stages. Different volatiles and different concentrations of volatiles can be associated with different stages of ripeness. Thus, the models can be trained to compare and correlate concentrations of each detected volatile with expected concentration ranges of volatiles associated with different ripening stages of the agricultural products.

FIG. 2 is an example apparatus for collecting volatiles of agricultural products to determine quality of the agricultural products. The apparatus depicted in FIG. 2 is the collection device 106A of FIG. 1 (e.g., also refer to FIG. 10).

The collection device 106A can include a container 200 (e.g., enclosure) and a tube 202. The container 200 can be sized to fit one agricultural product, such as the produce 108A depicted. The container 200 can also be sized to fit multiple agricultural products. For example, the container 200 can be a bin or other enclosure that contains a bunch of avocados. The bunch of avocados can be shipped together, from a same farm/vendor and/or to a same end-consumer retail environment. In some implementations, the bunch of avocados can be from a different farm/vendor.

The tube 202 can include a sorbent material 206, such as TENAX (e.g., refer to FIG. 10). In some implementations, the sorbent material 206 can be one or more other porous polymers such as PoraPak N and/or PoraPak Q. The sorbent material 206 can also be a graphitized Carbon, such as Carbograph 1TD, Carbopack B, Carbotrap B, Carbopack Y, and Carbotrap Y. The sorbent material 206 can collect volatiles as they are outgassed by the produce 108A. A portion of the tube 202 having the sorbent material 206 can be inserted into the container 200. The tube 202 can be filled with the sorbent material 206 and packed down with a plunger. The sorbent material 206 can also be placed along any portion of an interior of the tube 202.

Volatiles 208 from the produce 108A can flow up through a valve 204 of the tube 202 and collect on the sorbent material 206. Gases that are expired from the produce 108A can flow out through the tube 202. The volatiles 208 can naturally flow through the tube. The volatiles 208 can also be forced through the tube 202, such as with a fan unit 1006 (e.g., refer to FIG. 10). Any other air source or blower unit can also be positioned within the container 200 and used to assist in directing flow of outgassed volatiles 208 towards the sorbent material 206 in the tube 202. In some implementations, the container 200 can have both an inlet and an outlet, for example where gases flow through the tube 202. In some implementations, the container 200 can have just an inlet, where, for example, a pump is used to pump gases through the container.

As described herein, the sorbent material 206 can remain within the container for 30 to 60 minutes. Different timeframes can be used depending on the produce 108A. For example, more collection time can be used for testing one produce 108A since one produce 108A can outgas fewer volatiles than multiple produce. Less time can be used for collecting volatiles from a batch of produce since there can be more outgassed volatiles that can be collected at a given time. As another example, use of the fan unit 1006 can decrease a collection timeframe. The fan unit 1006 can increase airflow and direct that airflow towards the tube 202 and the sorbent material 206 therein. Thus, the volatiles 208 can collect more quickly on the sorbent material 206 with use of the fan unit 1006 or a similar air source or blower unit.

Once the collection timeframe is completed, the tube 202, having the sorbent material 206, can be removed from the container 200. The tube 202 may not have to be sealed once removed from the container 200 since the volatiles 208 are trapped on the sorbent material 206 and can only be removed when heated to a certain temperature threshold. As described in reference to FIGS. 1A-1C, the analysis computer system 102 can perform principal component analysis of the volatiles 208 to then map the volatiles 208 to different quality characteristics.

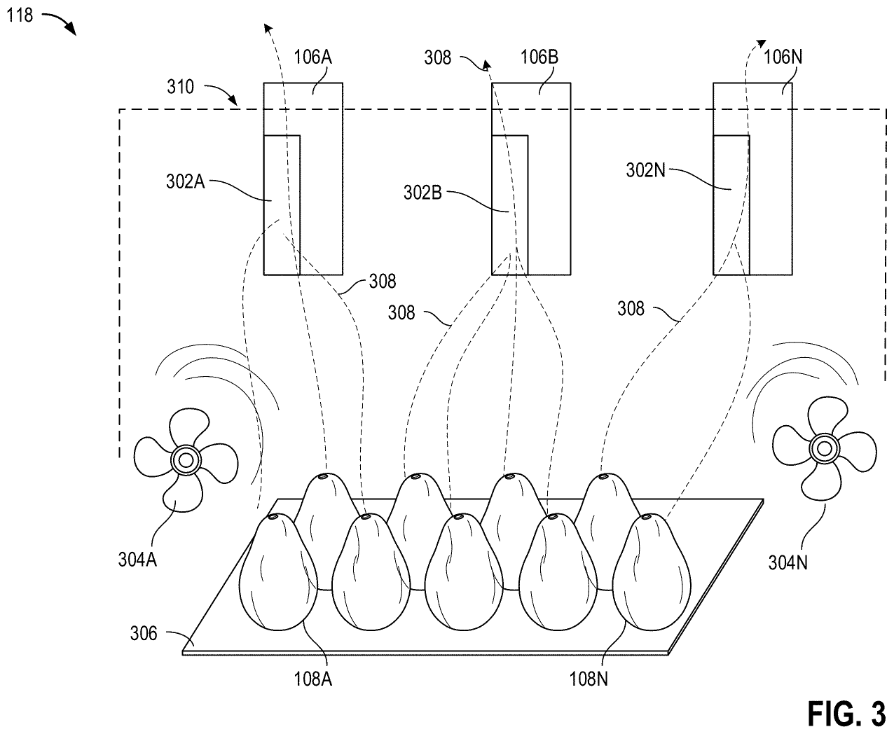

FIG. 3 is an example system for collecting volatiles of agricultural products to determine quality of the agricultural products. Agricultural products, such as produce 108B-N can be placed in an environment 118 once at a distribution center or other storage facility. The environment 118 can be an area or room for storing the produce 108B-N. For example, the environment 118 can be a storage room, a refrigeration room, a storage container, a shipping container, or a ripening room. The environment 118 can store the produce 108B-N that are all of a same type and packaged or otherwise bundled together on a pallet 306. For example, the environment 118 can store a batch or bulk of avocados on the pallet 306. Additional pallets, bins, and/or containers can be placed within the environment 118 to store batches of other agricultural products.

Collection devices 106B-N can be positioned throughout the environment 118 and configured to collect volatiles 308 that are released from the produce 108B-N. The collection devices 106A-N can be moved around the environment 118 to locations of different pallets of agricultural products. The locations can be enclosed area 310. The enclosed area 310 can be a space within the environment 118 where the pallet 306 of produce 108B-N is located. The enclosed area 310 may not include walls or a physical structure. Instead, the enclosed area 310 can be a portion of the environment 118 where the collection devices 106A-N are configured to specifically target capture of the volatiles 308 outgassed from a particular batch of agricultural products. In this example, the collection devices 106A-N are positioned in an enclosed area 310 above the pallet 306. Therefore, the collection devices 106A-N can collect the volatiles 308 that are outgassed from the produce 108B-N. Once the collection timeframe is complete and the volatiles 308 on sorbent material 302A-N are processed and analyzed, the collection devices 106A-N can be moved to another enclosed area 310. At the other enclosed area 310, the collection devices 106A-N can collect volatiles that are outgassed from another batch of agricultural products.

In some implementations, pipes can be extended from the collection devices 106A-N into each of the multiple bins or pallets 306 that are stored in the environment 118. The pipes can be configured to collect volatiles that are released in each of the bins or pallets 306. As a result, the collection devices 106A-N can be configured to capture the volatiles of different batches of agricultural products at a same time. This can be advantageous to quickly assess quality characteristics (e.g., ripeness) of each of the batches rather than assessing quality characteristics of each batch in series.

Additionally, the environment 118 can include fans 304A-N to assist in directing air flow throughout the environment 118. In some implementations, the fans 304A-N can be moved/positioned within the enclosed area 310. In some implementations, the fans 304A-N can be in fluid communication with the enclosed area 310 via one or more apertures, apparatus, pipes, and/or tubes that can be routed into the enclosed area 310. For example, the enclosed area 310 can include an aperture between the ambient environment 118 and gas contained within the enclosed area 310. The fans 304A-N can be in fluid communication with the aperture. The fans 304A-N can provide a positive pressure flow of gas from the ambient environment 118 into the enclosed area 310 that directs volatiles 308 outgassed by the produce 108A-N towards and through the collection devices 106A-N.

In some implementations, the fans 304A-N can be positioned in various locations throughout the environment 118 rather than being moved to the designated enclosed area 310. The fans 304A-N can direct or otherwise circulate ambient air around the enclosed area 310. The fans 304A-N can be positioned such that the ambient air is moved in a direction of the collection devices 106A-N. For example, the fans 304A-N can be configured to direct volatiles 308 that are outgassed from the produce 108B-N towards the collection devices 106A-N. By using the fans 304A-N, the volatiles 308 can be captured on the sorbent materials 302A-N of the collection devices 106A-N at a faster rate than if the fans 304A-N are not used in the environment 118. In some implementations, a pressurized gas supply can be configured to and in fluid communication with the enclosed area 310. The pressurized gas supply can be configured similarly to the fans 304A-N described herein.

The configuration of the environment 118 as depicted in FIG. 3 can be advantageous to determine quality characteristics, such as a current stage of ripeness, of the batch of produce 108B-N at any time. This determination can be made on demand and continuously, such that action can be taken as soon as the produce 108B-N is determined to be at a particular ripeness stage or exhibiting a particular quality characteristic. For example, by continuously monitoring and assessing outgassed volatiles, supply chain modifications can be dynamically made to ensure that the produce 108B-N does not go to waste. As an illustrative example, it can be preferred to ship avocados when they are at stage 2 ripeness so that they arrive at an end-consumer retail environment (e.g., grocery store) by a time they reach stage 3 ripeness. As soon as the avocados, in the environment 118, are detected to be reaching or at stage 2, supply chain modifications can be made to immediately ship the avocados to the end-consumer retail environment. Thus, the avocados can be at peak ripeness once provided to consumers for purchase at the end-consumer retail environment.

The configuration of the environment 118 can also be advantageous to eliminate need for human workers to complete destructive, labor-intensive testing on some of the produce 108B-N in order to determine quality characteristics of the produce 108B-N as an aggregate. In other words, human workers may not be required to puncture, squeeze, cut into, open, peel, or otherwise slice any of the produce 108B-N to determine whether the batch of the produce 108B-N is ready to be shipped to the end-consumer retail environment. Instead, human workers can move the collection devices 106A-N into different enclosed areas 310 within the environment 118 to target collection of volatiles from different batches of produce. Since the collection devices 106A-N can be moved to different enclosed areas 310, the human workers may not have to move pallets or bins that contain the batches of produce to access those batches of produce and manually test them. Worker efficiency can therefore improve.

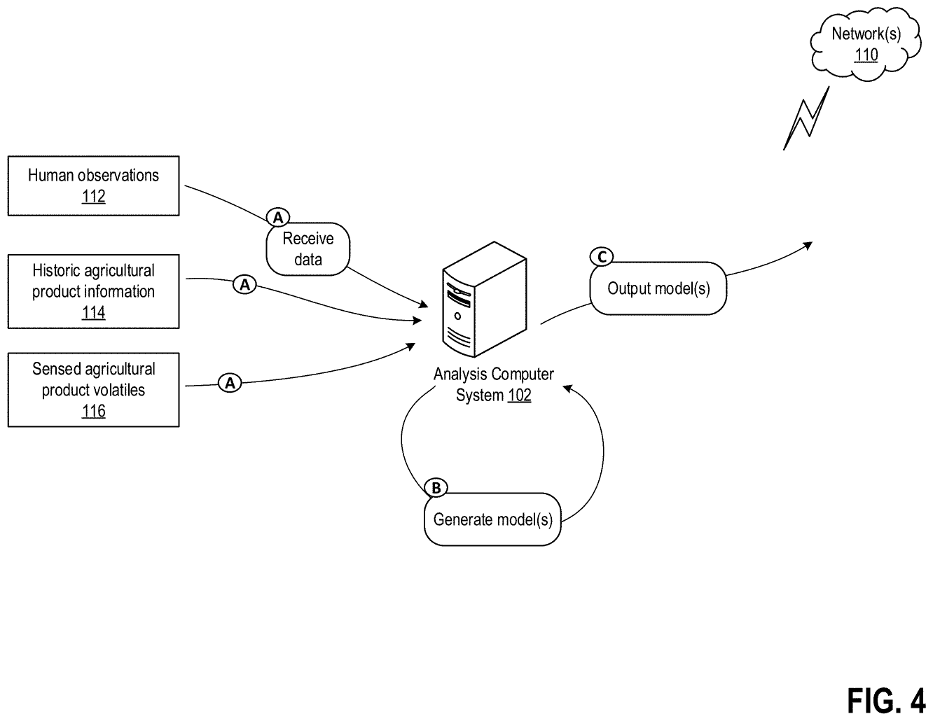

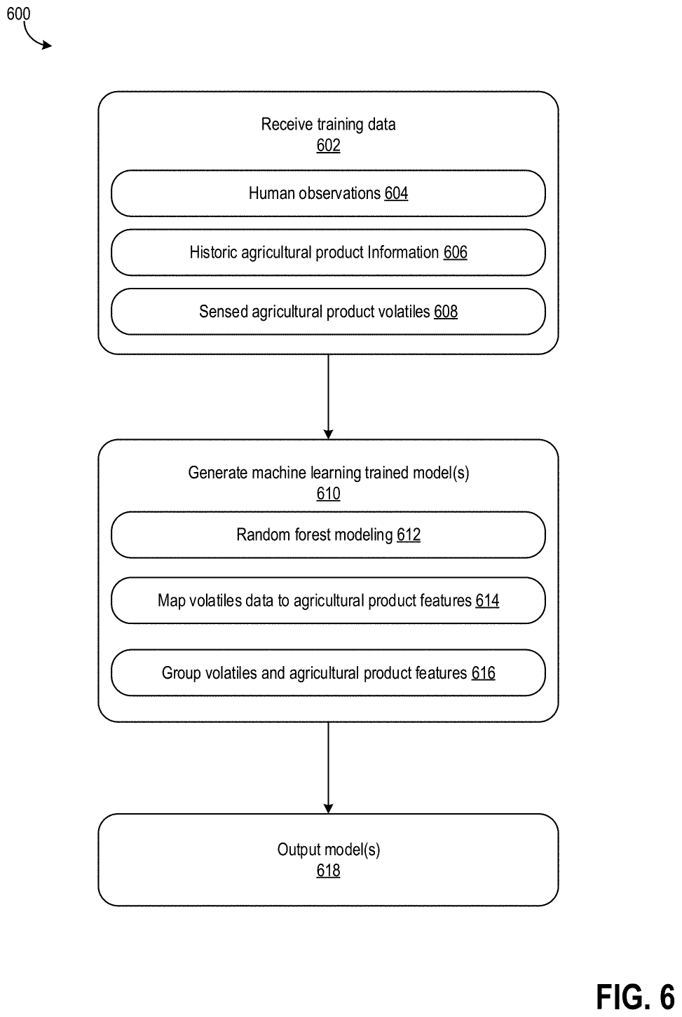

FIG. 4 is a conceptual diagram for generating a trained model that can be used for determining agricultural product quality (e.g., refer to FIG. 6). The analysis computer system 102 can receive data (step A), such as human observations 112, historic agricultural product information 114, and sensed agricultural product volatiles 116.

The human observations 112 can include destructive and/or non-destructive measurements of agricultural products that are taken by humans, such as workers in a distribution center. For example, the human observations 112 can include penetrometer or durometer data. The human observations 112 can also include indications of how firm or soft the products were to touch, how the products smelled, colors of the products, etc. The historic agricultural product information 114 can include place of origin of the agricultural products, times at which the agricultural product is expected to or has ripened, times at which the product is expected to or has been fresh, storage conditions, ripening conditions, shipping conditions, etc. The sensed agricultural product volatiles 116 can include time series strings of data of volatiles that have been captured for agricultural products using techniques described herein.

Using the received data, the analysis computer system 102 can generate one or more ripeness models (step B). The models can be generated using machine learning techniques and algorithms, such as convolution neural networks (CNNs). The models can be trained to correlate different concentrations of volatiles with different stages of ripeness, based on a type of the agricultural product. Each model can be generated to determine stages of ripeness for different types of agricultural products. The models can be trained to identify associations between different concentrations of the sensed volatiles 116 with ripeness stages based on qualities or features of the agricultural products that, according to the human observations 112, indicate the different ripeness stages. The models can also be trained to identify associations between different concentrations of the volatiles 116 with ripeness stages based on historic agricultural product information 114, such as historic ripening stages and conditions.

The analysis computer system 102 can then output the ripeness model(s) (step C). The outputted ripeness model(s) can be used during run-time to determine ripeness stages of one or more agricultural products.

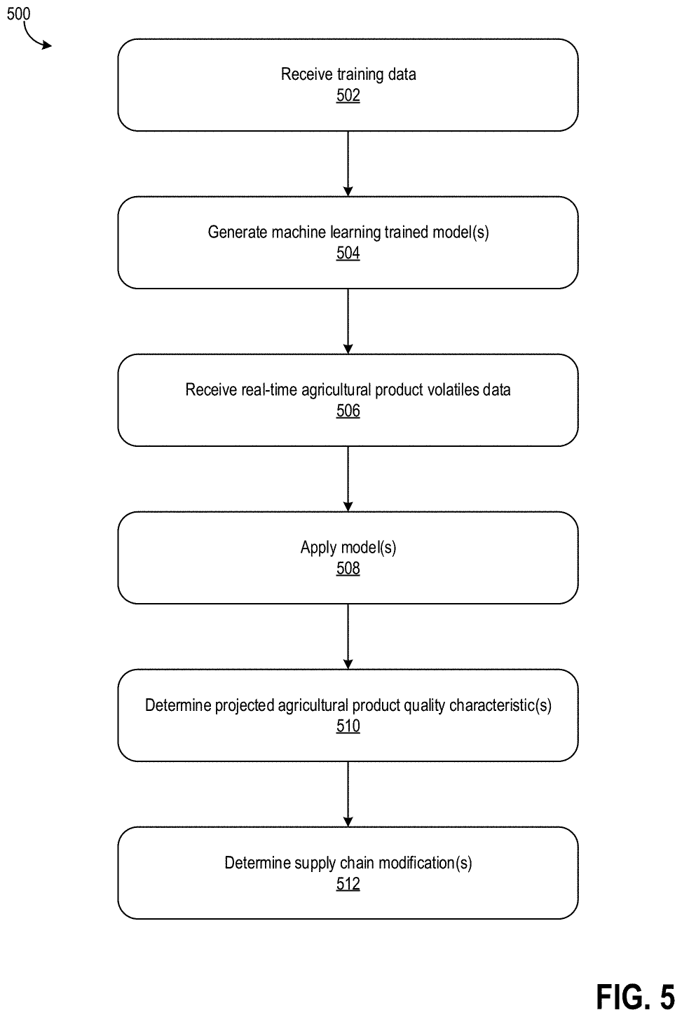

FIG. 5 is a flowchart of a process 500 for determining agricultural product quality. One or more blocks of the process 500 can be performed by the analysis computer system 102 and/or the supply chain computer system 104 depicted in FIG. 1A. The process 500 can also be performed by any one or more other computing systems. For simplicity and illustrative purposes, the process 500 is described from a perspective of a computer system.

Referring to the process 500, the computer system can receive training data in 502. As described in reference to FIG. 4, the training data can include human observations, historical information about the agricultural products, and sensed volatiles for the agricultural products. The training data can be retrieved from one or more data stores. The training data can also be received from one or more devices, computers, and/or systems, such as a gas chromatograph or a user device.

Using the training data, the computer system can generate machine learning trained model(s) in 504. The models can be generated using the techniques described herein, such as in reference to FIGS. 4 and 6. Models can be generated for each type of agricultural product. Models can also be generated based on different types of volatiles that are expected to be outgassed from different types of agricultural products. Moreover, models can be generated based on different concentrations of volatiles and/or classifications or groupings of volatiles. The models can be generated to correlate volatiles with different ripeness stages, as described herein.

The computer system can receive real-time agricultural product volatiles data in 506. The volatiles data can be received using the techniques described herein, as described in reference to FIGS. 1A-1B, 2, and 3. The volatiles can be received at a time that is later than and different than the time the computer system receives training data (502) and generates the models (504).

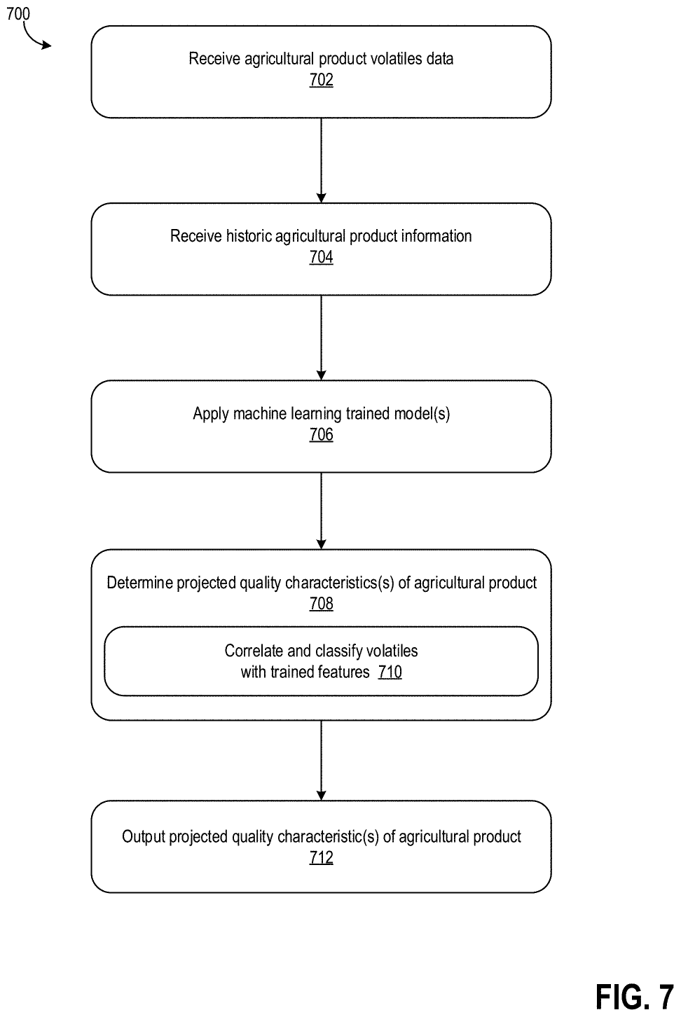

The computer system can then apply the model(s) to the volatiles data in 508. The models can be applied to the volatiles data in order to determine a current ripeness stage of the agricultural products whose volatiles have been collected. As described throughout this disclosure (e.g., refer to FIG. 7), the computer system can select one or more machine learning trained models to apply to the volatiles data. The models can be selected based on a type of the agricultural product, concentrations of volatiles in the volatiles data, and what volatiles appear in the volatiles data.

Next, the computer system can determine projected quality characteristic(s) of the agricultural product in 510. Such a determination can be made based on applying the models to the volatiles data. For example, one or more models can be trained to identify a current ripeness stage of the agricultural products. One or more models can also be trained to identify quality features or characteristics of the agricultural products based on the volatiles that are collected and concentrations of such volatiles. In some implementations, some concentrations of particular types of volatiles or groups of volatiles can indicate a taste, sweetness, or tartness of the agricultural products. Some concentrations of particular types of volatiles or groups of volatiles can indicate an acidity level of the agricultural products. Some concentrations of particular types of volatiles or groups of volatiles can indicate a softness or firmness of the agricultural products. Thus, using the techniques described herein, volatile data can be analyzed in order to non-destructively detect numerous conditions and characteristics about agricultural products that can impact the supply chain.

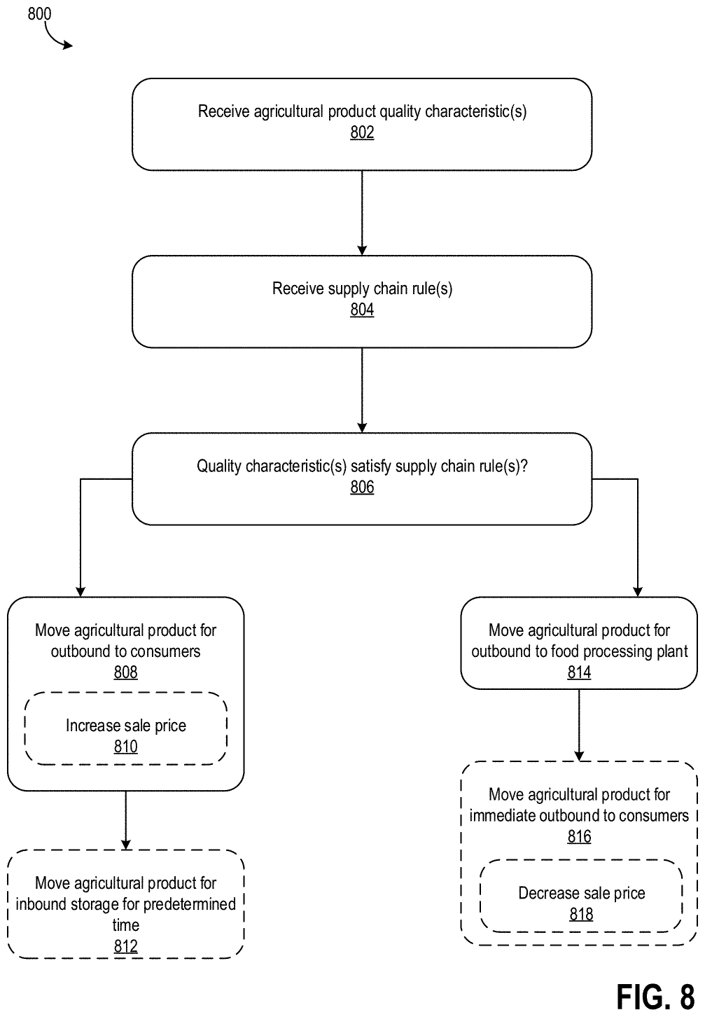

Accordingly, the computer system can determine one or more supply chain modifications in 512. As described herein, for example in reference to FIG. 8, supply chain modifications can be determined based on the current ripeness stage of the agricultural products. This can be advantageous to ensure that the agricultural products do not go to waste and that they can be delivered to end consumers at peak ripeness or otherwise when they reach a preferred/desirable quality for consumer consumption. In some implementations, supply chain modifications can be dynamically generated and/or updated as volatiles data is continuously captured, processed, and analyzed by the computer system.

As described in reference to FIG. 3, volatiles data can be continuously collected in some implementations. As a result the process 500 can be repeated in a feedback loop while the volatiles data is continuously collect. This continuous loop can be advantageous to detect when the ripeness stage or other quality characteristic(s) of the agricultural products changes to make dynamic and appropriate supply chain modifications. The continuous loop can therefore be advantageous to prevent or otherwise mitigate product-based waste.

FIG. 6 is a flowchart of a process 600 for generating a trained model that can be used for determining agricultural product quality. The process 600 can be performed by the analysis computer system 102 depicted in FIG. 1. The process 600 can also be performed by any one or more other computing systems. For simplicity and illustrative purposes, the process 600 is described from a perspective of a computer system.

Referring to the process 600, the computer system can receive training data in 602. As described herein (e.g., refer to FIG. 4), the training data can include human observations 604, historic agricultural product information 606, and sensed agricultural product volatiles 608. The human observations 604 can include features of the agricultural product that a human identified as indicative of different stages of ripeness for that agricultural product. The human observations 604 can be made at any time throughout the supply chain, between shipping the agricultural product from a place of origin to stocking shelves in a grocery store with the agricultural product. The historic agricultural product information 606 can include attributes about the agricultural product that can indicate ripeness stages or other quality characteristics of that product.

The sensed agricultural product volatiles 608 can be volatiles that have been collected over time for the agricultural product. The volatiles can be collected at different times throughout the supply chain and used to map or correlate this volatiles data with expected ripeness stages or other quality characteristics of the agricultural product. For example, volatiles can be collected before the product is transported from farm to distribution center, during transit to the distribution center, upon arrival at the distribution center, while being stored in the distribution center, during transit from the distribution center to an end-consumer retail environment, and upon arrival at the end-consumer retail environment. This time series of volatiles data can be advantageous to train models to more accurately correlate volatiles data with different ripeness stages and/or quality characteristics.

The computer system can then generate one or more machine learning trained models using the received training data in 610. Principal component analysis and random forest modeling techniques can be used to process individual volatile components and correlate them with different ripeness stages and quality features. Thus, to generate the models, the computer system can perform random forest modeling (612). Using random forest modeling, or similar supervised learning techniques, the computer system can build out a way that the volatiles data is processed such that components of the volatiles data can be better aligned.

The computer system can then map volatiles data (e.g., the sensed agricultural product volatiles 608) to agricultural product features (614). The features can indicate different characteristics that are expected for the agricultural product during different ripeness stages. Thus, the volatile data can be associated with different features of the agricultural product.

The computer system can also group the volatiles data and agricultural product features (616). In some implementations, particular groupings of volatiles can indicate one or more ripening stages. The particular groupings of volatiles can also correspond to different features of the agricultural product that are indicative of different ripening stages. For example, volatiles data and agricultural product features can be grouped based on ripeness stages, internal qualities, vascular browning, chilling, stem rot, etc. Refer to Table 1 for a list of volatiles and concentrations of such volatiles that are assessed for different characteristics of agricultural products.

The computer system can then output the models for run-time use in 618. In some implementations, the models can be updated and/or improved using volatiles data that is collected during run-time use of such models. The models can also be updated and/or improved using outcomes of these models during run-time use.

FIG. 7 is a flowchart of a process 700 for using the trained models to determine agricultural product quality in real-time. The process 700 can be performed by the analysis computer system 102 depicted in FIG. 1. The process 700 can also be performed by any one or more other computing systems. For simplicity and illustrative purposes, the process 700 is described from a perspective of a computer system.

Referring to the process 700, the computer system can receive agricultural product volatiles data in 702. As described throughout this disclosure (e.g., refer to FIGS. 1A-C, 2, 3), volatiles can be collected on sorbent materials in collection devices that can be placed in containers or other types of enclosed areas. The volatiles can be desorbed from the material in a gas chromatograph such that the volatiles can be analyzed. Volatiles can be identified, their concentrations can be determined, and sometimes, the volatiles can be grouped into classifications. This data can then be received by the computer system and used in the process 700. The volatiles data can also be in a time series.

The computer system can also receive historic agricultural product information in 704. The block 704 can also be performed at a same time as block 702 or before block 702. The historic information can include place of origin, historic ripening conditions, ripening stages of the agricultural product, shipping conditions, and any other information that can be collected about the agricultural product. As described herein, projected ripeness or other quality features of the agricultural product can depend on historical progression of the product, such as where the agricultural product was grown, how far it traveled to a distribution center, travel conditions, whether the product was coated in a ripening agent, etc.

The computer system can apply the machine learning trained model(s) to the volatiles data in 706. The model(s) can also be applied to the historic information. In some implementations, the historic information can be used to identify which model(s) to apply to the volatiles data. For example, the historic information can indicate that this particular agricultural product ripens slower and under different conditions than other agricultural products of a same type. This particular agricultural product can have a different ripening process because of its place of origin and/or shipping conditions, for example. Since this agricultural product ripeness slower and under different conditions, the computer system can select and apply a ripeness model that is specific to this particular agricultural product rather than general to all agricultural products of the same type.

Once the model(s) is applied to the volatiles data, the computer system can determine projected quality characteristics of the agricultural product in 708. For example, the model(s) can be trained to correlate and classify volatiles in the volatiles data with product features (710). Concentrations of particular types of volatiles can correlate to one or more features of the agricultural product that indicate quality and/or ripeness stages of that product. Refer to Table 1 for examples of volatiles and concentrations of such volatiles that can be analyzed using the techniques described herein. Example quality characteristics that can be modeled and identified from the volatiles data include ripeness, firmness, rot, taste, sweetness, acidity, etc. One or more models can also be trained to group the volatiles into classes and then correlate those classes with particular agricultural product features and/or ripeness stages. For example, one or models can be trained to determine flavor, off-flavor development, mold, rot, and other features from the volatiles. By determining the projected quality characteristics of the agricultural product, the computer system can predict a current quality of ripeness stage of the agricultural product in a non-destructive way.

The computer system can then output the projected quality characteristics of the agricultural product (712). In other words, the computer system can provide the projected quality characteristics (e.g., ripeness stage) to a user device or another computing system. Relevant stakeholders in the supply chain can view this output and make decisions about how to move the agricultural product throughout the supply chain (e.g., refer to FIG. 8). The computer system can also store the projected quality characteristics in a data store. This output can then be used by the computer system to train and improve one or more models.

FIG. 8 is a flowchart of a process 800 for determining supply chain modifications based on determined agricultural product quality. The process 800 can be performed by the supply chain computer system 104 depicted in FIG. 1. The process 800 can also be performed by any one or more other computing systems. For simplicity and illustrative purposes, the process 800 is described from a perspective of a computer system.

Referring to the process 800, the computer system can receive agricultural product quality characteristics in 802. The quality characteristics can be received from the analysis computer system 102. The quality characteristics can also be retrieved from a data store.

The computer system can also receive supply chain rules in 804. The supply chain rules can be received from a computer system, server, data store, and/or user device. For example, a relevant stakeholder can view the output of the quality characteristics (e.g., refer to FIG. 7) and decide that supply chain modifications should be made. The quality characteristics can indicate that the particular agricultural product is already at peak ripeness. Thus, the stakeholder can decide that the agricultural product should be delivered to end-consumers immediately. The stakeholder can therefore select supply chain rules that are associated with shipping agricultural products and provide those rules to the computer system. The stakeholder can select the rules at a user device and the user device can transmit them to the computer system.

In some implementations, the computer system can automatically determine which supply chain rules to select. This determination can be based on the quality characteristics of the agricultural product. Thus, if the agricultural product is projected to be at peak ripeness, the computer system can automatically select one or more supply chain rules that relate to procedures that can be followed to deal with a product at peak ripeness.

The supply chain rules can indicate steps that can be taken to move the agricultural product throughout the supply chain. The rules can correlate to different conditions and quality characteristics. For example, a rule can establish that if an agricultural product is at peak ripeness, it should be shipped to an end-consumer retail environment that is geographically closest to a current location of the agricultural product. Another rule can establish that if the product is past peak ripeness, it should be shipped to a food processing plant. Yet another rule can establish that if the product is not yet ripe, the product can be moved into storage in the distribution center. As another example, another rule can establish that if the product is not yet ripe, the product can be shipped to an end-consumer retail environment that is geographically farthest away from the current location of the agricultural product. One or more other supply chain rules can exist.

The computer system can determine whether the quality characteristics of the agricultural product satisfy the supply chain rules in 806. An example supply chain rule in this example of FIG. 8 can be whether the agricultural product is reaching peak ripeness or whether the product has passed peak ripeness. Another example supply chain rule can be whether the agricultural product is free from mold, internal rot, desiccation, or another poor quality feature. Yet another example supply chain rule can be whether the agricultural product has a normal or expected sweetness/taste. Another example supply chain rule can be whether the agricultural product has a normal or expected smell or odor.

Here, if the computer system determines that the quality characteristics of the agricultural product satisfy the supply chain rule(s), then the computer system can generate instructions that cause the agricultural product to be moved for outbound to consumers in 808. In other words, the computer system can determine that the agricultural product has desirable quality characteristics that consumers expect in their products. The computer system can decide that the product should still be shipped to its intended end-consumer retail environment. In some implementations, the computer system can determine that the product should be shipped to an end-consumer retail environment that is geographically farther away from the current location of the agricultural product. The computer system can compare quality characteristics of the agricultural product to those of other agricultural products of a same type to make a decision of which products should be sent to which end-consumer retail environments.

The computer system can also optionally increase a sale price of the particular product since the product is of higher or more desirable quality (810). In the example supply chain rules mentioned above, the block 808 can be performed if the agricultural product (i) is reaching peak ripeness, (ii) does not have any signs of mold, internal rot, desiccation, or other poor quality features, and/or (iii) has normal or expected sweetness/taste and/or smell. In some implementations, the computer system may not increase the sale price, but because the product is a higher or more desirable quality, consumer satisfaction can increase, which can result in an increase in sales.

Optionally, the computer system can also generate instructions that cause the agricultural product to be moved to inbound storage for a predetermined period of time (812). This supply chain modification can be preferred when the agricultural product is deemed to not yet be ripe and/or to have a certain amount of days before ripening begins or the agricultural product reaches peak ripeness. Therefore, it can be desirable to store the product for a longer period of time before shipping it to consumers.

Returning to block 806, if the computer system determines that the quality characteristics of the agricultural product do not satisfy the supply chain rule(s), then the computer system can generate instructions that cause the agricultural product to be moved for outbound shipment to a food processing plant in 814. In the example supply chain rules mentioned above, the block 814 can be performed if the agricultural product (i) has passed peak ripeness, (ii) has signs or a certain quantity of mold, internal rot, desiccation, or other poor quality features, and/or does not have the normal or expected sweetness/taste. In other words, the agricultural product may not be in a desirable condition to be purchased by consumers or consumed. Thus, the supply chain can be modified for this agricultural product by moving the product to the food processing plant. As an example, the computer system can determine that the agricultural product presently does not have rot but will be rotting within a certain number of days. If the certain number of days is less than an amount of time that it would take to ship the agricultural product to an end-consumer retail environment, then the computer system can determine that moving the product to a food processing plant instead is more desirable. Moving poor quality products to food processing plants can be advantageous to avoid product-based waste.

Optionally, the computer system can determine that the agricultural product should instead be moved immediately to consumers in 816. For example, the computer system can determine that although the product is at peak ripeness, consumers may still desire to purchase the product and use it immediately. Therefore, the product can be shipped to an end-consumer retail environment. As described throughout this disclosure, the computer system can determine that the product should be sent to the end-consumer retail environment that is geographically closest to the current location of the product. After all, the product would be in transit for a short period of time, during which the quality of the product may not worsen. Moreover, the computer system can optionally decrease a sale price of the product (818) that is shipped immediately to consumers. Decreasing the sale price of the product can incentivize consumers to purchase the product, despite its lower quality. This incentive can be advantageous to avoid or otherwise mitigate product-based waste.

One or more other supply chain rules and modifications can be realized in the process 800. The supply chain rules and modifications can be determined by relevant stakeholders in the supply chain. Moreover, the rules and modifications can be specific to different types of agricultural products, quality characteristics, ripeness stages, vendors, consumer expectations, and/or end-consumer retail environments.

FIG. 9 is a schematic diagram of an exemplary GCxGC set up 900. The setup 900 can be used to detect volatile markers of different agricultural products. As an example, the setup 900 can be used to detect volatile markers of avocados. For illustrative purposes, the setup 900 is described in relation to detecting the volatile markers of avocados. Using the setup 900, volatile molecules outgassed by an avocado can first be extracted by solid phase microextraction (SPME) or thermal desorption (TD) followed by analysis by comprehensive two-dimensional gas chromatography (GCxGC). The GCxGC setup 900 can be selected as a separation technique in this exemplary embodiment as it provides sufficient separation capacity and captures a wide variety of compounds without interferences.

Referring to the setup 900, a non-polar (e.g., polydimethylsiloxane) column 908 can be connected to a polar (e.g., polyethyleneglycol) column 910 by 2 capillary flow plates 902 and 904 that are connected by a modulation loop 912. Carrier gas flow can be controlled through an inlet 914 and a second pneumatically controlled modulator 906 (PCM). The second column 910 and restrictor 916 can be connected to front (FID 1) and back (FID 2) detectors 918 and 920 of the GC setup 900.

A locally fabricated flow modulator can be used in a reverse fill/flush format. The microfluidic plates 902 and 904 can be connected by the deactivated fused silica modulation loop 912 (11 cm×0.530 mm i.d.). The carrier gas, hydrogen, can be delivered to the plates 902 and 904 by the PCM 906 and flow switching can be achieved through a three-port miniature solenoid valve. Modulation periods of 5 seconds can be achieved with this flow modulator, which can be a maximum time that maintains a minimum of 3 cuts per peak.

GC×GC separations can be performed using a non-polar stationary phase in the first dimension (e.g., column 908) and a polar stationary phase in the second dimension (e.g., column 910). Column 908 can be selected to be a polydimethylsiloxane phase (DB-1msUI, 20 m×0.180 mm i.d.×0.18 μm film thickness), while column 910 can be a polyethylene glycol phase (DB-WAXmsUI, 5 m×0.250 mm i.d.×0.25 μm film thickness). Gas flows can be operated at constant flow of 0.350 mL/min in column 908, supplied by the inlet 914, and 22 mL/min through column 910 can be supplied through the PCM 906. A 3 m×0.100 mm i.d. deactivated fused silica tube can act as a bleed line for reversed fill/flush modulation and can be connected to the second FID 920.

FIG. 10 depicts an exemplary sample collection apparatus 1000 for collecting volatiles of agricultural products. The apparatus 1000 can include a container 1002 (e.g., 32 oz wide mouth jar), a brushless miniature fan 1006, and a rubber septum 1004. An agricultural product, such as avocado 108A, can be placed in the container 1002 with the fan 1006 spinning. The apparatus 1000 can also include a tube 1008 having a fiber 1010 (e.g., sorbent material). For adsorption of volatile molecules, the fiber 1010 can be inserted through the rubber septum 1004 where the fiber 1010 can be exposed for 30 min. The fiber 1010 can be exposed for different time periods. The exposure time period can depend on a quantity of agricultural products in the container 1002, a size of the agricultural product(s), a size/power of the fan 1006, a size of the fiber 1010, and/or one or more additional factors. Desorption can take place in a GC inlet.

SPME can be preformed using a manual field unit with the fiber 1010. Sampling by TD can be performed using the tube 1004. The tube 1004 can be a stainless-steel TD tube packed with a universal sorbent material (e.g., the fiber 1010; Markes International, PN: C3-AXXX-5266), and a desorption unit. In some implementations, an additional ¼″ and ⅛″ bulkhead union can be used to connect the TD tube 1004 and a nitrogen purge line, respectively.

The fiber 1010 using in SPME can be a PDMS/Carboxen/DVB fiber. One or more other fibers can be used with the apparatus 1000. The PDMS/Carboxen/DVB fiber 1010 can be selected since it can extract a wide range of compounds from agricultural products, such as the avocado 108A. By placing the avocado 108A into the container 1002, volatiles can be outgassed and contained, which can then be absorbed onto the SPME fiber 1010. Using the brushless miniature fan 1006, absorption can occur for 30 minutes. Where the fan 1006 is not used, absorption can occur over 60 minutes. The extracted compounds on the fiber 1010 can then be analyzed by a GCxGC (e.g., refer to the GC setup 900 in FIG. 9).

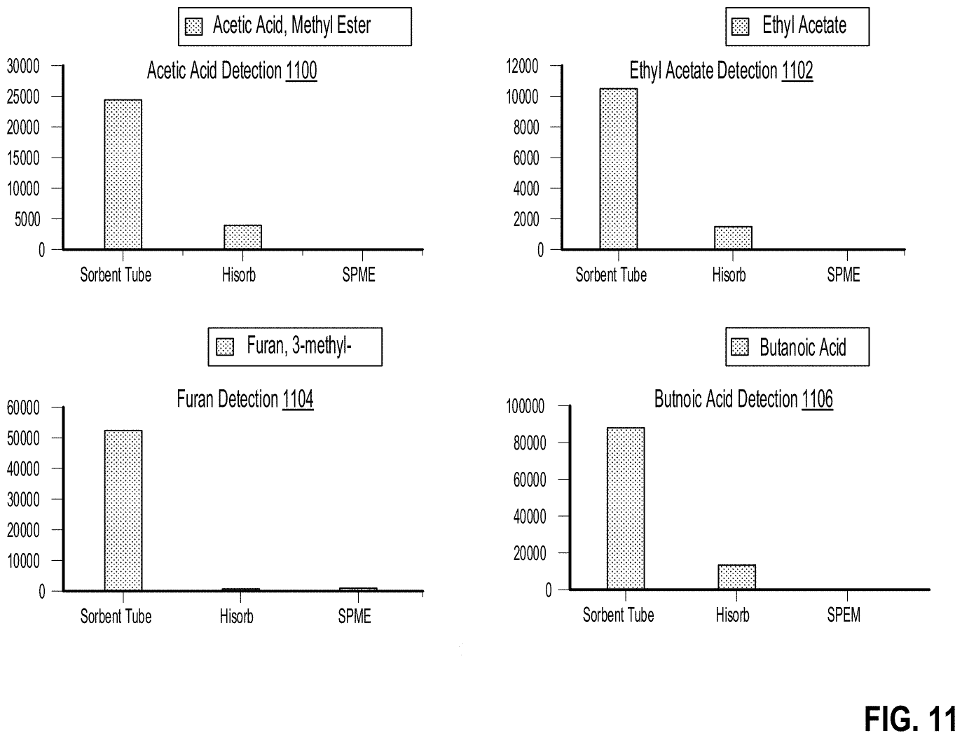

FIG. 11 is a graphical depiction summarizing an exemplary volatile sample collection comparison of TD, SPME, and Hisorb. Hisorb is similar to SPME with a larger capacity. Desorption can be performed with a Centri TDU and can be analyzed by a ThermoFisher GC-MS system. Area counts of acetic acid, ethyl acetate, furan and butanoic acid can be recorded and plotted on a y-axis in graphs 1100, 1102, 1104, and 1106, respectively. Thus, TD sampling can be compared to SPME for detectability of 4 different identified compounds collected from agricultural products such as avocados. Volatiles can be collected for 30 minutes with each different sampling technique. From this comparison, it can be observed that detectability of these compounds can increase using TD sampling (e.g., refer to the apparatus 1000 in FIG. 10).

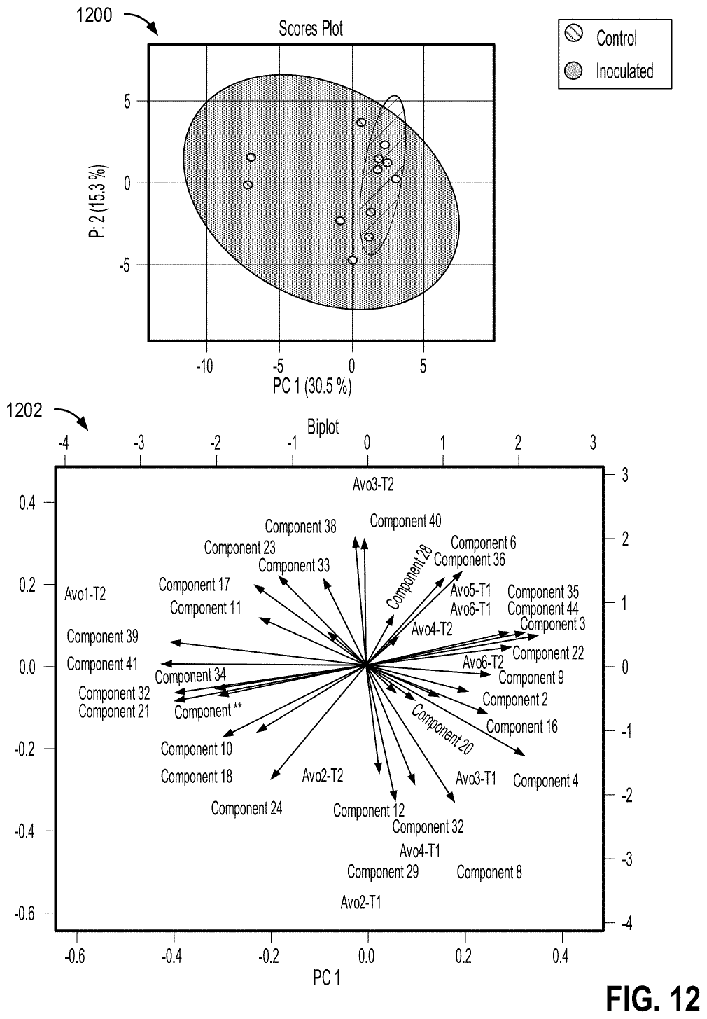

FIG. 12 depicts an exemplary Scores plot 1200 and Biplot 1202 of two principle components to demonstrate how measured components relate to agricultural products. As an illustrative example, the plots 1200 and 1202 relate to measured components of 12 tested avocados. Components 6, 22, 28, 35, 36, and 45 (among others) appear to be related to test avocados that are inoculated with a causative agent of a test infection, such as stem rot.

In this example, three avocados were inoculated with 1000 spores of C. gloeosporioides in 10 uL of water and three other avocados were injected with 10 uL of water as a control. A day after inoculation, volatiles off-gassed by the avocados were measured by SPME-GCxGC as described above. This procedure was repeated over 2 days. The resulting control and inoculated chromatograms were compared and 42 common components that spanned the volatile compound profile were integrated. The resulting areas were tabulated and preliminary statistical analysis was performed to determine trends of results.

Principle Component Analysis (PCA) was performed with the resulting scores as depicted in the biplot 1202. By looking at the first two principle components, the relationship of each component that was integrated to the others can be measured. The inoculated avocados were found to have similarities, and their outgassed volatiles lie in a similar space in the scores plot 1200. Further from the biplot 1202, the components that can be most significant to that avocado can be determined. As volatile components of the inoculated avocados fall into the same region, components 6, 22, 28, 35, 36, 45, and others can indicate stem end rot infections induced in these avocados, and are characteristic volatile markers associated with this infection. The biplot 1202 therefore can indicate which volatile components drive groupings of quality features for an agricultural product, such as avocados. A magnitude of vectors shown in the biplot 1202 can indicate significance of the components when classifying the agricultural product.

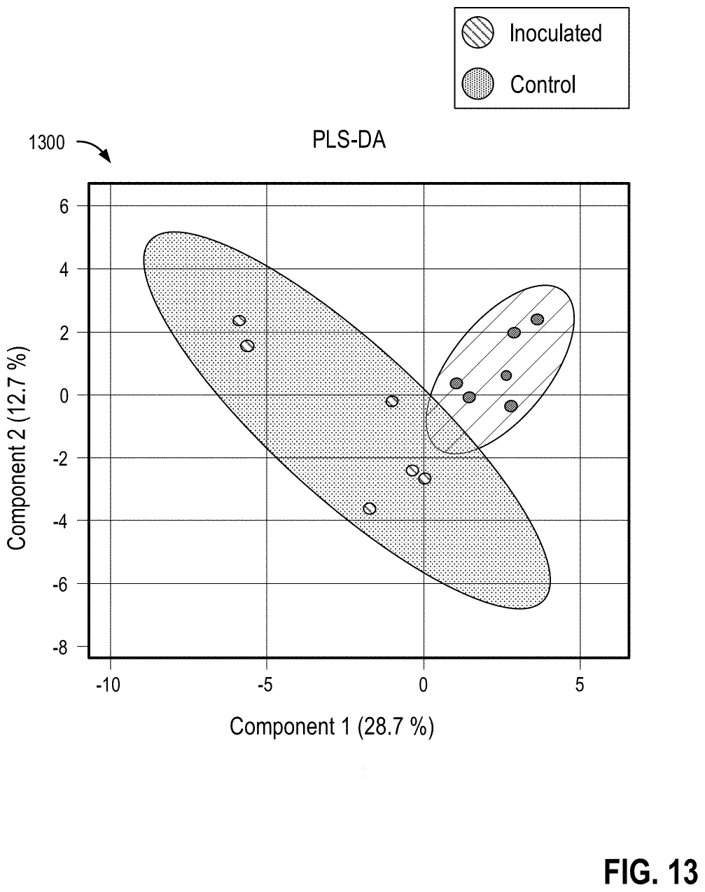

FIG. 13 depicts Partial Least Squares-Discriminant Analysis (PLS-DA) results 1300 using groups labeled control and inoculated agricultural products. As described in reference to FIG. 12, for illustrative purposes, avocados can be tested. One sample set can include 6 avocadoes per group. Two distinct groups of avocados can be identified. To produce predictive models, supervised analytics such as PLS-DA can be applied. The plot 1300 demonstrates using SPME-GCxGC to help produce a predictive model to diagnose latent infections in agricultural products such as avocados. By combining TD with GCxGC and Time of Flight Mass Spectrometry (ToFMS), some specific volatile markers associated with infection caused by C. Gloeosporioides can be more easily identified. The identities of the volatile markers can then be determined by analyzing the volatile markers from the TD experiments with GCxGC ToFMS.

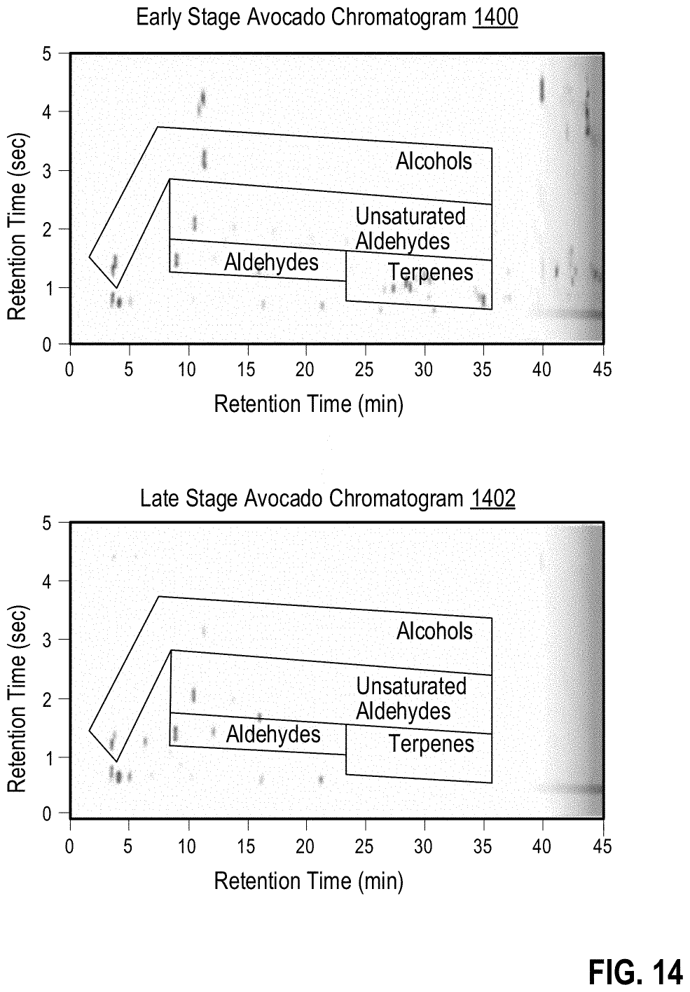

FIG. 14 depicts a representative chromatogram of test agricultural products. For illustrative purposes, as described in reference to FIGS. 10-13, the test agricultural products can be avocados. FIG. 14 depicts an early stage (e.g., 88 shore) avocado GCxGC chromatogram 1400 and a late stage (e.g., 45 shore) avocado GCxGC chromatogram 1402. Template of group types that are detected are overlaid showing terpenes, saturated and unsaturated aldehydes, and alcohols. The late stage chromatogram 1402 depicts significantly less compounds in comparison to the early stage chromatogram 1400 where the unsaturated aldehydes and terpenes are most abundant.

The illustrative, test avocados depicted in FIG. 14 are Mexican Hass avocados. These avocados can be initially measured by firmness using a durometer. A firmness measurement of 60 shore can correspond to an edible stage that a consumer would find at a supermarket or similar stakeholder. For flavor profiling, the test avocados were cut to remove pulp, which was weighed and blended with an equal amount of water. This blended mixture was placed into a 20 mL headspace vial where a known amount of internal standard (n-hexane) was added. The vial was then crimped closed for use in microextraction of outgassed volatiles.

SPME was performed using a manual field unit with a PDMS/Carboxen/DVB fiber (e.g., refer to the apparatus 1000 in FIG. 10). An optimal extraction procedure in this exemplary process was heating the avocado sample to 85° C. for 30 minutes while the fiber was exposed to the headspace. The collected volatiles were then desorbed in the inlet of the GC for 5 minutes at 270° C. After the 5 minute desorption, the resulting chromatogram was blank, indicating that all compounds absorbed by the fiber had been desorbed into the GC.

Multiple avocado samples treated as described above were analyzed by SPME-GC-MS to identify compounds related to aroma and flavor. The most abundant compounds identified included alcohols (hexanol, penten-3-ol), aldehydes (hexanal, nonanal, trans-2-hexenal, and trans-2-nonenal), and terpenes (limonene and myrcene). However, in analyzing the GC-MS chromatogram, co-elution was observed, resulting in partial positive detection of unknown compounds (match scores of the unknown compounds were between 600-700; full positive detection corresponds to a match score of 1000). Based on these initial findings, reference standards were used to encompass these chemical classes to determine an elution profile by GCx GC. This produced well-defined, ordered chromatograms and allowed templates to be made that distinguished different classes of compounds.

Referring to the FIG. 14, at the early maturity stage 1400 (e.g., with a firmness of 88 shore), a highest number of volatile compounds was observed. With a large number of compounds present, the possibility of co-elution is more likely; however, with increased separation power from GCxGC, this possibility can be reduced. From a study of 6 avocados at this early stage 1400, an average of 70 compounds were observed, with a maximum number of resolved compounds of greater than 100. The difference between the number of compounds observed is likely due to biological variances among the avocados tested.

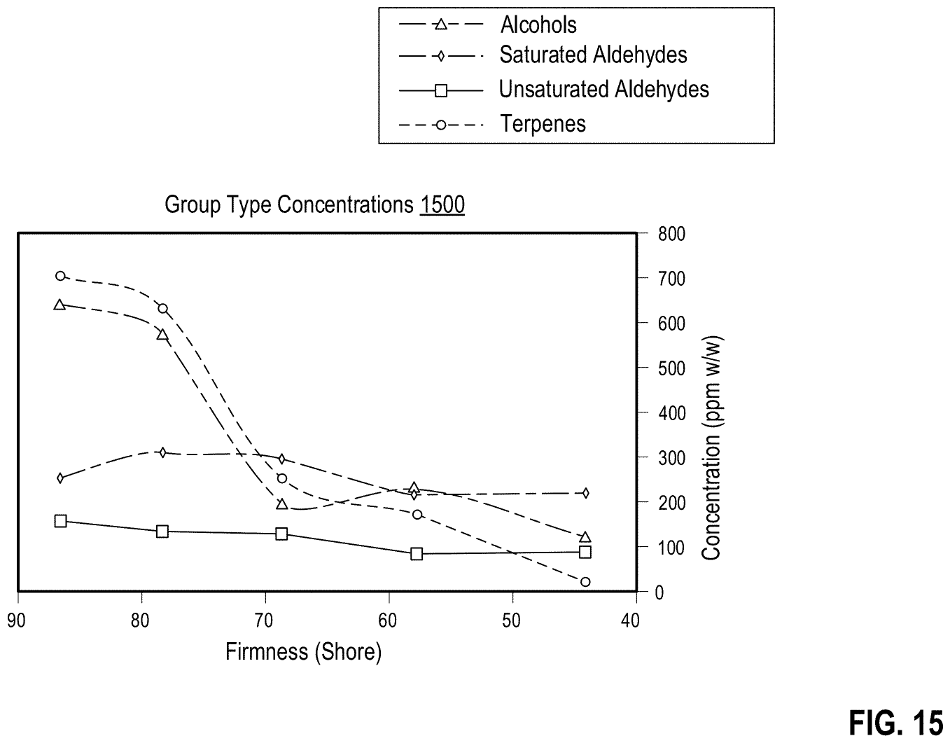

FIG. 15 is a graphical depiction representing average concentration of group types 1500 over a lifetime of an example avocado. As the avocado ages and becomes softer, a general trend of decreasing concentrations of flavor compounds can be observed when quantifying based on group types. The output depicted in the graph 1500 can be used to predict shelf life of the agricultural product and other quality information about such product.

For illustrative purposes, FIG. 15 is described in reference to quantitative analysis of flavor compounds in test avocados (e.g., refer to FIG. 14). For example, while GCxGC can provide an informative qualitative chromatogram, flame ionization detectors (FID) can be used for quantitative analysis and data collection. To determine reliability and robustness of the described method, figures of merit were determined in this illustrative example. To account for variability in sample extractions, as well as in avocado samples, an internal standard was added to an avocado mixture. The internal standard was chosen based on its absence in avocados as well as similarity in carbon number and volatility to primary compounds of interest. A solution of n-hexane in water was prepared at a final concentration of 0.2 ppm w/w.

To demonstrate repeatability in quantification of extracted compounds, a single avocado was prepared and separated into 5 headspace vials, each with a same amount of internal standard added. Three compounds, an early-, mid-, and late-eluting peak, were selected to measure peak area. This resulted in an average % RSD of 2.2%. These were analyzed over a span of <8 hours, and subsequently all avocados were prepared and analyzed within this timeframe.

Response factors were determined by obtaining calibration curves for 1-hexanol, trans-2-hexenal, and heptadienal over a concentration range from 0.1 to 10 ppm w/w. The resulting response factors were determined to be 0.0287, 0.0268, and 0.0244 concentration/area, respectively. These compounds were selected as they span various classes of interest and are of particular interest in avocado volatile analysis. By determining the response factor of these species, an average can be used and applied to all species in the resulting chromatograms. To test the effectiveness of this response factor, a spike of hexanal at 1 ppm w/w was added to an avocado sample. The recovery was then observed to be 94.7% using the average response factor of 0.0266 concentration/area.

As described herein, analyzing compound classes as opposed to individual compounds can be advantageous to determine trends representative of avocado ripening processes. In this illustrative example, a sample set of 50 avocados showed similarly inconclusive trends when looking at individual compounds. Table 1 displays calculated concentrations of selected flavor compounds identified using reference standards, as well as calculated concentrations for classes of compounds (e.g., alcohols, saturated aldehydes, unsaturated aldehydes, and terpenes). Trends can be harder to observe as biological variance becomes more pronounced. For example, % RSD for a most prominent compound, hexanal, can range from 17-52% when looking at different stages of ripeness. This variability can be attributed to biological variance, which can result in different individual compounds making up a composition of oil in avocados. This variability of individual compounds in avocados can be overcome using GC xGC. As individual component analysis can vary, the use of GCxGC has an added advantage of quantifying classes of compounds based on elution time (e.g., all alcohols will have a similar elution time). Thus, quantifying by classes of compounds as opposed to individual compounds can be beneficial to normalize biological variability.

TABLE 1
Concentration of identified flavor compounds and group
types found at various stages of ripeness for avocados.

| | Concentration (ppm w/w) | | | | |
|---|---|---|---|---|---|
| | Avg | Avg | Avg | Avg | Avg |
| | Firmness | Firmness | Firmness | Firmness | Firmness |
| | (Shore) | (Shore) | (Shore) | (Shore) | (Shore) |
| | 86.7 | 78.3 | 68.8 | 57.9 | 44.3 |
| Compound | (n = 6) | (n = 15) | (n = 9) | (n = 12) | (n = 9) |
| 1-penten-3- | BDL | BDL | BDL | 0.16 | 0.18 |
| ol | | | | | |
| 1-penten-3- | 0.48 | BDL | BDL | BDL | 0.22 |
| one | | | | | |
| Hexanal | 161.98 | 239.04 | 231.88 | 163.29 | 175.32 |
| Hexanol | 423.22 | 463.62 | 102.89 | 48.16 | 22.56 |
| Limonene | 2.07 | 0.80 | 1.41 | 0.93 | 0.19 |
| Methyl | BDL | 0.20 | BDL | BDL | BDL |
| Hexanoate | | | | | |
| Myrcene | BDL | 0.43 | 0.03 | BDL | BDL |
| Nonanal | 11.60 | 7.98 | 10.18 | 4.56 | 5.23 |
| t,t-2,4- | 22.00 | 17.35 | 22.19 | 3.28 | 0.41 |
| Hexadienal | | | | | |
| t-2-Hexenal | 99.89 | 86.56 | 79.12 | 36.23 | 65.41 |
| t-2-Nonenal | 3.40 | 0.66 | 2.12 | 0.08 | 0.26 |
| Alcohols | 642.65 | 571.69 | 199.42 | 224.03 | 123.25 |
| Saturated | 253.07 | 309.04 | 295.48 | 218.45 | 218.03 |
| Aldehydes | | | | | |
| Unsaturated | 155.81 | 133.63 | 126.87 | 83.17 | 88.11 |
| Aldehydes | | | | | |
| Terpenes | 702.67 | 632.74 | 250.56 | 169.74 | 20.08 |

The major classes of compounds that were observed in the GC×GC chromatogram include terpenes, saturated and unsaturated aldehydes, and alcohols. By quantifying the total compounds in these regions, relative standard deviations can be improved by at least a factor of 2. For example, hexanal, as previously discussed, lies in a region of saturated aldehydes, which has a maximum % RSD of 16. With improved quantification within these groups, trends become more apparent, as seen in FIG. 15. A decrease can occur in both the alcohol and terpene groups, whereas the saturated and unsaturated aldehydes can remain relatively constant, with just a slight decrease in abundance throughout maturation.

Thus, by classifying and quantifying based on group type, biological variability can be reduced. Regardless of oil content, byproducts that form most of the aroma compounds can be quantified by group in the GC×GC chromatogram, allowing for determination of trends that are representative of ripeness. More accurate ripening volatile marker timelines can then be generated based on these trends.

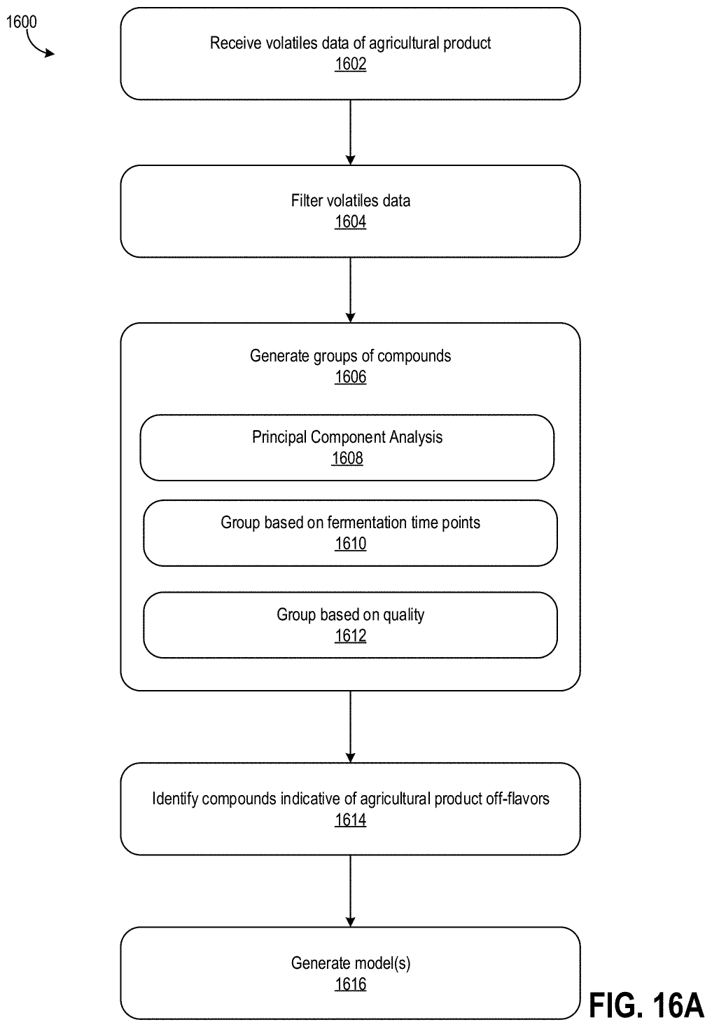

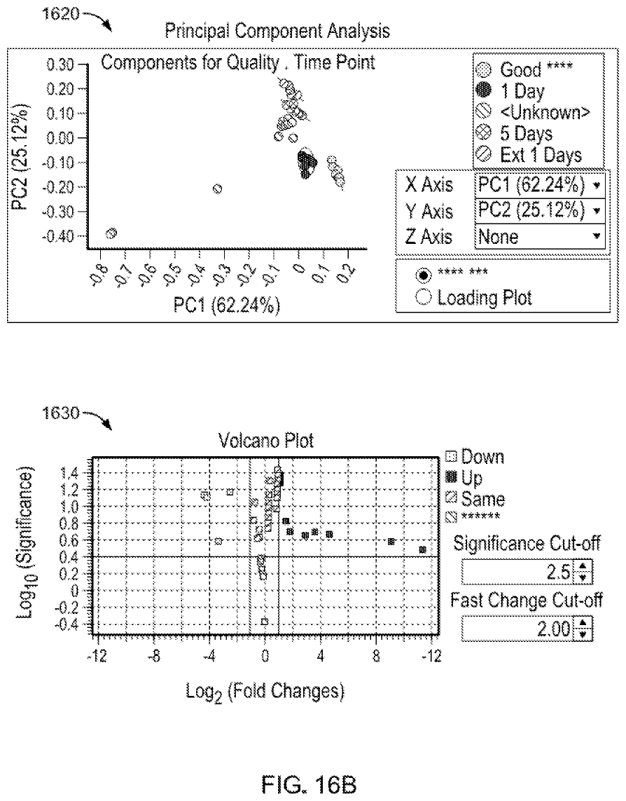

FIG. 16A is a flowchart of a process 1600 for analyzing low abundance volatiles that can be outgassed by example mandarins. FIG. 16B shows graphical depictions of identified volatiles that are outgassed by the example mandarins in FIG. 16A. The process 1600 can be performed by the analysis computer system 102 depicted in FIG. 1. The process 1600 can also be performed by any one or more other computing systems. Moreover, performing the process 1600 can include use of an SPME-GX×GC-MS to capture lower abundance volatile compounds that can be outgassed by agricultural products. For simplicity and illustrative purposes, the process 1600 is described from a perspective of a computer system.

Referring to the process 1600 in FIG. 16A, the computer system can receive volatiles data of an agricultural product in 1602. In the example of FIGS. 16A-B, the agricultural product is a mandarin. The agricultural product can be any other produce, including but not limited to fruits such as mangoes. The volatiles data can be received as described throughout this disclosure.

The computer system can filter the volatiles data in 1604. In other words, the volatiles data can undergo initial processing. Such filtering can include integrating and removing high background that may appear in one or more chromatograms that make up the volatiles data. As a result of filtering, lower abundance volatile compounds can be more apparent and used or further analysis.

In 1606, the computer system can generate groups of compounds based on the filtered volatiles data. After all, a combination of volatile compounds, including volatile compounds in low abundance, can contribute to off-flavors of agricultural products. As part of generating groups of compounds, the computer system can perform principal component analysis in 1608. Principal component analysis can also be used to identify important features that can indicate off-flavors development in the agricultural product. The computer system can group the compounds based on fermentation time points (1610). The computer system can also group compounds based on quality (1612).

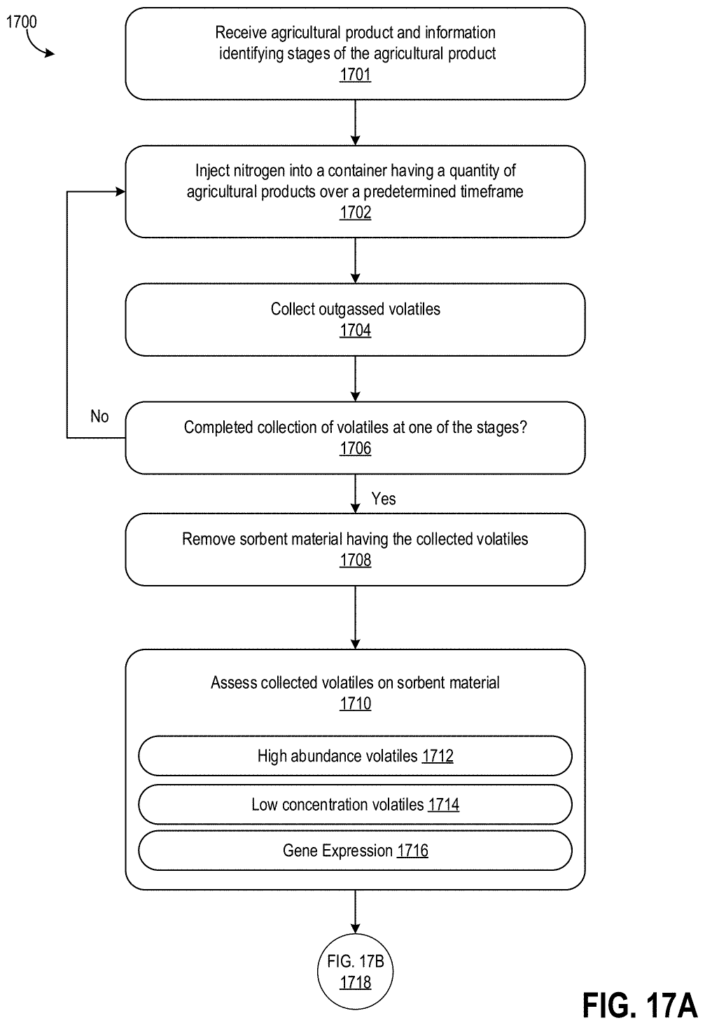

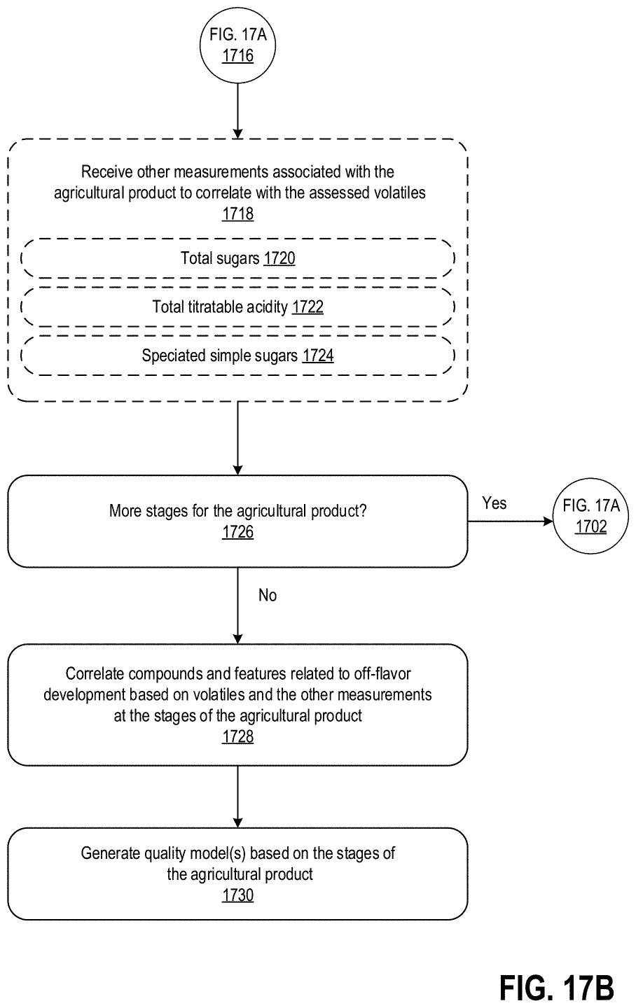

Next, the computer system can identify compounds indicative of agricultural product off-flavors in 1614. As shown in FIG. 16B, clear groups of compounds can be identified from performing principal component analysis in 1608. As shown in principal component analysis graph 1620, clear groups of compounds are seen between different time points throughout a fermentation process (refer to FIGS. 17A-B).

When observing volcano plot 1630 to determine which features have a greatest impact on these groupings, and therefore the fermentation process, 9 key features can be observed. In the illustrative example of mandarins in FIGS. 16A-B, the key compounds that were down-regulated and decrease with fermentation, are mainly terpene compounds: Alpha-pinene, limonene, linalool, germacrene D, and beta-farnesene. With respect to flavor, as these mandarins decrease in terpenes, they can start to lose flavor, as opposed to developing off-flavors. Conversely, when looking at up-regulated compounds, those that increase with fermentation, ethanol and ethyl acetate were found to be key compounds of interest. Initially, ethanol was shown to be low in concentration and as the mandarins fermented, other ethanol-derived compounds, such as ethyl acetate, were formed. The combination of the up- and down-regulated compounds can be useful in determining models for the fermentation process, ultimately leading to prediction of fermentation or off-flavors of agricultural products.

Referring back to the process 1600 in FIG. 16A, the computer system can also generate model(s) in 1616. The models, as described throughout this disclosure, can be generated using machine learning techniques. The models can be trained to identify groups of identified compounds or features of interest in volatiles data. The models can also be trained to correlate the identified groups with one or more off-flavors, quality, and/or time points in the fermentation process. Thus, quality information about agricultural products can also be determined from low abundance volatile compounds. As mentioned above, the process 1600 can be used to identify low abundance volatile compounds indicative of off-flavors in a variety of different agricultural products. For example, models can be generated based on low abundance volatile compounds that are analyzed in mangoes, mandarins, other fruits, and vegetables. One or more models can be generated for each type of agricultural product.

FIGS. 17A-B is a flowchart of a process 1700 for identifying volatile compounds that can be related to off-flavor development in agricultural products. The process 1700 can be used to determine which compounds and/or combinations of compounds can be related to off-flavor development in different types of agricultural products. Off-flavor development can vary between variety and category of agricultural products. The process 1700 can be used to characterize off-flavor sensitivity at a variety level. Moreover, when off-flavor sensitivity is analyzed from the variety level, determinations to treat the agricultural products can be more accurately made such that the agricultural products maintain optimal quality. Development of off-flavors can also impact consumer satisfaction and sales of the agricultural products. Being able to measure off-flavor development can be advantageous to determine when the agricultural products should be delivered to consumers to thereby ensure higher levels of satisfaction and increased sales. In other words, if the consumers are happy about the products they purchase because they taste, smell, and/or feel to be peak ripeness, then the consumers may be more likely to purchase more of the products. As an illustrative example, if based on analysis of outgassed volatiles, mandarins are expected to create off-flavors within a certain number of days, one or more supply chain modifications can be made to move the mandarins to consumers in a shorter timeframe. Thus, the mandarins can be purchased and enjoyed by the consumers before they begin off-flavor development. Consumer satisfaction can be high, and the consumers may be more inclined to purchase more mandarins. Increased sales can also have an added benefit of reducing or otherwise avoiding agricultural product-based waste.

Sometimes, overtreating agricultural products can create off-flavors, which can lead to fermentation of such agricultural products. As a main off-flavor and compound developed during fermentation, ethanol content can be a quality metric that is assessed and tracked through volatiles data.

One or more blocks in the process 1700 can be performed by the analysis computer system 102 depicted in FIG. 1. One or more blocks in the process 1700 can also be performed by any one or more other computing systems. For simplicity and illustrative purposes, one or more blocks in the process 1700 are described from a perspective of a computer system.

Referring to the process 1700 in both FIGS. 17A-B, agricultural products and information about stages of the agricultural product can be received in 1701. For example, the agricultural products can be placed in a container, storage container, or other enclosed area for holding the products. The information can indicate checkpoints, times, stages, and/or degrees of a fermentation process for the particular received agricultural products. The information can also indicate, for example, different ripeness stages of the agricultural products. The received information can be used to determine when and for how long to collect volatiles of the agricultural products and/or when to assess collected volatiles associated with the agricultural products.

Nitrogen can also be injected into a container having a quantity of the agricultural products over a predetermined timeframe (1702). Injecting the container with nitrogen can create an anaerobic environment that causes the agricultural products to undergo a fermentation process. The predetermined timeframe can be a length of time that the agricultural products are projected to complete the fermentation process. The predetermined timeframe can also be a length of time that is desired to force the agricultural products to undergo the fermentation process. In some implementations, nitrogen can be injected into the container for 30 minutes at a flow of over approximately 300 mL/min. The flow rate can be based on a size of the container, a size of the agricultural products, and/or how many agricultural products are in the container.

As an illustrative example, the agricultural products can be mandarins. A combination of changes in volatile composition, fruit characteristics, and processing conditions can contribute to development of off-flavors in mandarins. One or more attributes can be taken into consideration when determining off-flavor characterization of the mandarins. Such attributes can include volatile compounds (e.g., ethanol, ethyl-esters, etc.), sugar to acid ratio, peel permeability, impact of storage time, impact of elevated temperatures, and impact of handling.

In particular, during postharvest storage or after exposure to anaerobic atmospheres, mandarins can develop off-flavors more rapidly than other citrus varieties, and the occurrence of these off-flavors can be associated with increases in juice ethanol and acetaldehyde (AA) levels. In fresh citrus fruit, a common alcohol is ethanol, which provides ethanol-like odor. Accumulation of ethanol can be associated with development of off-flavors in stored fruit. High levels of ethanol that accumulate during storage can also serve as substrates for subsequent downstream esterification reactions with acyl-CoAs, derived from fatty acid and amino acid catabolism. This can result in accumulation of ethyl-ester volatiles, which can cause over-ripeness and undesired odors.

The sugar to acid ratio can be measured as brix to acid or totally soluble solids to total acidity. This can be a quality metric that describes palatability of mandarins, such as a balance of sour and sweet. Sugar levels can remain relatively constant with a slight decrease in acidity. The sugar to acid ratio can experience minimal changes during cold storage or throughout a lifecycle of the mandarins.

As for peel permeability, peel of the mandarin can be less permeable to gas, and especially to ethanol vapors, than that of grapefruit or other fruits. Differences in evaporation properties of these volatile compounds, and not their catabolism rates, can play a role in governing accumulation of such volatile compounds in an internal atmosphere of the citrus fruit. For example, although a whole peel of ‘Star Ruby’ grapefruit can be thicker than that of ‘Murcott’ mandarins, a dense flavedo layer was thicker in the mandarins. The flavedo of mandarins can also contain more oil glands, which can be impermeable to gases, than that of grapefruit. An albedo layer in the former can also be thinner but can contain denser cells than that of the latter fruit. The flavedo portion of the peel can provide a main barrier to gas diffusion in citrus fruits like mandarins.

Time in cold storage can also influence quality of the mandarins. Similarly, elevated temperatures can increase volatiles. For example, at 68 F, volatiles associated with off-flavors can increase within 24 hours. On the other hand, in an intermediate temperature of 50 C, the mandarins may not show an increase in volatiles. Time in cold storage can therefore influence response as well as fruit maturity. Warm temperature storage can cause a loss in acidity in the mandarins.

Finally, changes in handling and storage can also impact quality and off-flavors of the mandarins. For example, ethanol can accumulate during storage and can be influenced by pack-line handling. Differences in aroma active compounds can be detected due to storage and handling, wherein 10 compounds can change significantly due to storage duration and 5 compounds can change due to handling.

Mandarin fruit quality can deteriorate in flavor and sensory acceptability, sometimes beginning only a few weeks after harvest, due to accumulation of off-flavor volatiles. Off-flavors can develop prior to external desiccation and can be a limiting factor in shelf life of mandarins. Thus, using the techniques described throughout this disclosure and in reference to FIG. 17, off-flavor production in mandarins can be measured, monitored, and assessed to ensure that good quality mandarins can be provided to end consumers, suppliers, and retailers. Moreover, the techniques described herein can be used to determine mandarin shelf-life extension(s) and quantify differences in seasonality, maturity, variety, and storage parameters. Mandarins are used as an example. The techniques described herein can also be applicable to other types of agricultural products, including but not limited to other fruits, such as grapefruits and mangoes. In this illustrative example, the mandarins can be placed in a container and assessed over a timeframe of 8 days. Nitrogen can continuously be flushed into the container over the course of the 8 days.

Still referring to the process 1700 in FIG. 17, outgassed volatiles can be collected in 1704. Collection of the volatiles can occur as described throughout this disclosure.

Next, the computer system can determine whether collection of volatiles completed at one of the stages for the agricultural product in 1706. This determination can be based on whether a checkpoint (e.g., time, stage in fermentation process, degree of fermentation, etc.) has been reached in 1706. For example, the checkpoint can include times, stages, and/or degrees during the fermentation process of the agricultural product. Example checkpoints can be time-based, such as 1 hour, 2 hours, 4 hours, 6 hours, and 24 hours after the fermentation process begins. One or more different checkpoints can also be used. For example, the checkpoints can be projected or determined stages of fermentation for the agricultural product. The checkpoint can also be based on one or more values that are sensed by sensors proximate to the agricultural product, such as oxygen and/or nitrogen sensors.

In the illustrative example of mandarins, collection of volatiles can be completed at multiple predetermined timeframes over 8 days. A first checkpoint when volatiles collection is complete can be when the mandarins are placed in the container, a second checkpoint can be 1 day after placement in the container and injection of nitrogen, a third checkpoint can be 2 days after placement in the container, a fourth checkpoint can be 5 days after placement in the container, and a fifth checkpoint can be 8 days after placement in the container (e.g., the last day in the predetermined timeframe). In some implementations, each of these checkpoints or times at which collection of volatiles are completed can be based on quantities of nitrogen that are sensed in the container.

If the collection of volatiles has not been reached, the process 1700 can return to block 1702. Nitrogen can continue to be flushed into the container. The computer system can repeat blocks 1702-1706 until the collection of volatiles has been completed.

If the collection of volatiles is completed in 1706, then a sorbent material having the collected volatiles can be removed such that the volatiles can be processed and analyzed in 1708. The computer system can perform a suite of analytical tests on the collected volatiles, as described below. As a result, the computer system can identify volatile compounds and/or combinations of compounds that, at that particular stage, degree, and/or time in the fermentation process, indicate off-flavor development and/or other quality characteristics of the agricultural product.

In the illustrative example of mandarins, at each of the 5 time checkpoints, the computer system can analyze 10 sample collections of volatiles data. The assessments described below can be performed for each of the 10 sample collections. The assessments can also be tracked through each of the 5 time checkpoints during the 8 day fermentation process.

Accordingly, the computer system can assess the collected volatiles on the sorbent material in 1710. The collected volatiles can be filtered and processed at each of the checkpoints at which volatiles collection is completed. For example, as described above, volatiles can be collected at each stage of the fermentation process. The collected volatiles can then be assessed for each stage. As part of filtering and processing the collected volatiles, ion 44 can be extracted and excluded from additional assessments described below. Title sum integration can also be performed on the collected volatiles before performing the additional assessments.

Assessment can be performed as described throughout this disclosure. The computer system can analyze high abundance volatiles (e.g., using HS-GC-FID) in 1712. The computer system can analyze low concentration volatiles (e.g., using SPME-GC×GC-MS) in 1714 (e.g., refer to FIGS. 16A-B). As described throughout this disclosure, volatiles can account for much of a sensory aspect of agricultural products, such as mandarins.

In the illustrative example of mandarins, 3 main volatiles related to fermentation, ethanol, acetaldehyde, and ethyl acetate, can be quantified and tracked using HS-GC-FID during the fermentation process of 8 days. The computer system can determine that ethanol levels may rise significantly while the mandarins undergo the fermentation process. For example, a concentration of ethanol can range from 13.1-398.7 ppm before the mandarins are placed in the container. After 9 days without oxygen, concentration of ethanol can spike to 11,183.4-26,533.2 ppm. Additionally, acetaldehyde can start to increase as the mandarins ferment. After 5 days (e.g., the fourth time checkpoint), ethyl derivatives can start to arise, specifically ethyl acetate.

In addition to testing and assessing these higher concentration volatiles using HS-GC-FID, a simultaneous technique can be used to analyze volatiles in lower abundance, as described in reference to FIGS. 16A-B. SPME-GCxGC-MS can be used to capture lower abundance compounds as well as to provide identification of such volatiles. These volatiles can then be analyzed by the computer system using the techniques described herein (e.g., refer to FIGS. 16A-B). Finally, the computer system can analyze gene expression (e.g., PCR) of the agricultural products in 1716.

In some implementations, the computer system can optionally receive other measurements associated with the agricultural product to correlate with the assessed volatiles in 1718. These other measurements can include sugar levels, acidity, and speciated simple sugars. Thus, the computer system can analyze total sugars (e.g., brix) in 1720, thereby correlating the total sugars to the assessed volatiles. The computer system can also analyze total titratable acidity (e.g., acid/base titration) in 1722. The computer system can analyze speciated simple sugars (e.g., LC-ELSD) in 1724.

In the illustrative example of mandarins, the computer system can assess brix and acid values of the mandarins at each of the 5 checkpoints over the 8 days of the fermentation process. The computer system can determine that during fermentation, sugars may not change too much while total acidity can decrease slightly after 8 days. Generally, brix and acid values can be reported as a ratio corresponding to a sweet/tart ratio in the agricultural product. When looking at this ratio throughout the fermentation process, a slight increase can be seen due to a slight decrease in acid as the agricultural product undergoes fermentation. The computer system can determine that brix and acid values can be more commonly used for determining readiness for agricultural products to undergo harvest. With this in mind, brix and acid can be tested on first time checkpoints to determine an initial state of the agricultural product.

Moreover, in this illustrative example of mandarins, simple sugars can be speciated and tracked throughout the fermentation process. Fructose, glucose, and sucrose can be monitored over the 8 days. The computer system can identify, for example, a slight decrease in these monitored sugars since they were metabolized by the agricultural product during fermentation. Thus, the speciated sugars can be used to identify onslaught of fermentation and/or different stages of fermentation in agricultural products

Next, the computer system can determine whether there are any additional stages of the agricultural product of which volatiles are collected and analyzed (1726). In the illustrative example of mandarins, the computer system can determine whether volatiles have been assessed at the first, second, third, fourth, and fifth time checkpoints during the 8 days of the fermentation process.

If there are additional stages, the computer system can return to block 1702. Blocks 1702-1726 can be repeated until there are no more remaining stages to assess volatiles associated with the agricultural product. In the illustrative example of mandarins, the blocks 1702-1726 can be repeated until the fermentation process of 8 days is complete.

If there are no more remaining stages of the agricultural product (e.g., the fermentation process is complete), the computer system can correlate compounds and features related to off-flavor-development based on volatiles and the other measurements at the stages of the agricultural product in 1728. Thus, the computer system can use the data, statistics, and chemometric analysis to pull out important features to identify compounds of interest related to off-flavor development. The computer system can compare the assessments that were made at each stage of the agricultural product during the fermentation process to identify the key compounds and features. The computer system can identify the key compounds and features associated with each stage where collection of volatiles was completed and correlate those compounds and features to stages in the fermentation process. The computer system can also identify the key compounds and features associated with the fermentation process in the aggregate. After all, volatile analysis can produce significant changes amongst initial and various stages during the fermentation process.

The computer system can generate quality model(s) based on stages of the agricultural product in 1730. Models can be generated as described throughout this disclosure. The models can be generated based on the features that were identified in 1728 and their corresponding volatiles compounds and/or combinations of compounds and other measurements. For example, assessment of brix and acid values can be used to generate models that are trained to identify an initial state of the agricultural product. Such models can be used to determine whether the agricultural product is ready to undergo harvest. As another example, assessment of speciated sugars can be used to generate models that are trained to identify when the agricultural product begins or is currently in a fermentation process. Thus, quality of the agricultural product can be assessed. As yet another example, assessment of high abundance volatiles as well as low concentration volatiles can be used to generate models that are trained to identify and/or predict fermentation, stages of fermentation, off-flavor development, and/or particular off-flavors of agricultural products.

As described throughout this disclosure, volatiles assessment can be performed for different types of agricultural products. As an illustrative example, mangoes can be tested using the process 1700. When volatiles are assessed for the mangoes in the process 1700, the computer system can determine that a mango can have less than 10 ppm of ethanol before being placed in the container. After 9 days in the container without oxygen and only nitrogen, the mango can have 13,350.9 ppm of ethanol. This information can be used to identify and model quality of mangoes based on abundance of certain volatile compounds that are outgassed by the mangoes. As another illustrative example, grapefruits can be tested using the process 1700. When the volatiles are assessed for the grapefruits in the process 1700, the computer system can determine that a grapefruit can have less than 200 ppm of ethanol before being placed in the container. After 6 days in the container without oxygen and with only nitrogen, the grapefruit can have almost 16,000 ppm of ethanol. This information can be used by the computer system to identify and model quality of grapefruits based on abundance of certain volatile compounds that are outgassed by the grapefruits.

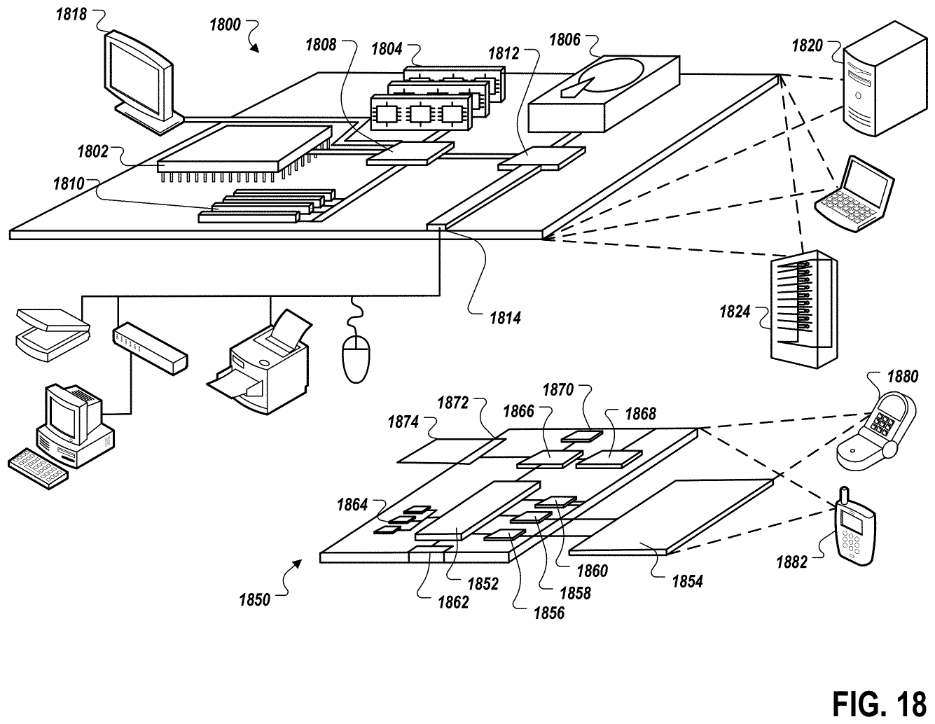

FIG. 18 shows an example of a computing device 1800 and an example of a mobile computing device that can be used to implement the techniques described here. The computing device 1800 is intended to represent various forms of digital computers, such as laptops, desktops, workstations, personal digital assistants, servers, blade servers, mainframes, and other appropriate computers. The mobile computing device is intended to represent various forms of mobile devices, such as personal digital assistants, cellular telephones, smart-phones, and other similar computing devices. The components shown here, their connections and relationships, and their functions, are meant to be exemplary only, and are not meant to limit implementations of the inventions described and/or claimed in this document.

The computing device 1800 includes a processor 1802, a memory 1804, a storage device 1806, a high-speed interface 1808 connecting to the memory 1804 and multiple high-speed expansion ports 1810, and a low-speed interface 1812 connecting to a low-speed expansion port 1814 and the storage device 1806. Each of the processor 1802, the memory 1804, the storage device 1806, the high-speed interface 1808, the high-speed expansion ports 1810, and the low-speed interface 1812, are interconnected using various busses, and can be mounted on a common motherboard or in other manners as appropriate. The processor 1802 can process instructions for execution within the computing device 1800, including instructions stored in the memory 1804 or on the storage device 1806 to display graphical information for a GUI on an external input/output device, such as a display 1816 coupled to the high-speed interface 1808. In other implementations, multiple processors and/or multiple buses can be used, as appropriate, along with multiple memories and types of memory. Also, multiple computing devices can be connected, with each device providing portions of the necessary operations (e.g., as a server bank, a group of blade servers, or a multi-processor system).

The memory 1804 stores information within the computing device 1800. In some implementations, the memory 1804 is a volatile memory unit or units. In some implementations, the memory 1804 is a non-volatile memory unit or units. The memory 1804 can also be another form of computer-readable medium, such as a magnetic or optical disk.

The storage device 1806 is capable of providing mass storage for the computing device 1800. In some implementations, the storage device 1806 can be or contain a computer-readable medium, such as a floppy disk device, a hard disk device, an optical disk device, or a tape device, a flash memory or other similar solid state memory device, or an array of devices, including devices in a storage area network or other configurations. A computer program product can be tangibly embodied in an information carrier. The computer program product can also contain instructions that, when executed, perform one or more methods, such as those described above. The computer program product can also be tangibly embodied in a computer- or machine-readable medium, such as the memory 1804, the storage device 1806, or memory on the processor 1802.

The high-speed interface 1808 manages bandwidth-intensive operations for the computing device 1800, while the low-speed interface 1812 manages lower bandwidth-intensive operations. Such allocation of functions is exemplary only. In some implementations, the high-speed interface 1808 is coupled to the memory 1804, the display 1816 (e.g., through a graphics processor or accelerator), and to the high-speed expansion ports 1810, which can accept various expansion cards (not shown). In the implementation, the low-speed interface 1812 is coupled to the storage device 1806 and the low-speed expansion port 1814. The low-speed expansion port 1814, which can include various communication ports (e.g., USB, Bluetooth, Ethernet, wireless Ethernet) can be coupled to one or more input/output devices, such as a keyboard, a pointing device, a scanner, or a networking device such as a switch or router, e.g., through a network adapter.

The computing device 1800 can be implemented in a number of different forms, as shown in the figure. For example, it can be implemented as a standard server 1820, or multiple times in a group of such servers. In addition, it can be implemented in a personal computer such as a laptop computer 1822. It can also be implemented as part of a rack server system 1824. Alternatively, components from the computing device 1800 can be combined with other components in a mobile device (not shown), such as a mobile computing device 1850. Each of such devices can contain one or more of the computing device 1800 and the mobile computing device 1850, and an entire system can be made up of multiple computing devices communicating with each other.

The mobile computing device 1850 includes a processor 1852, a memory 1864, an input/output device such as a display 1854, a communication interface 1866, and a transceiver 1868, among other components. The mobile computing device 1850 can also be provided with a storage device, such as a micro-drive or other device, to provide additional storage. Each of the processor 1852, the memory 1864, the display 1854, the communication interface 1866, and the transceiver 1868, are interconnected using various buses, and several of the components can be mounted on a common motherboard or in other manners as appropriate.

The processor 1852 can execute instructions within the mobile computing device 1850, including instructions stored in the memory 1864. The processor 1852 can be implemented as a chipset of chips that include separate and multiple analog and digital processors. The processor 1852 can provide, for example, for coordination of the other components of the mobile computing device 1850, such as control of user interfaces, applications run by the mobile computing device 1850, and wireless communication by the mobile computing device 1850.

The processor 1852 can communicate with a user through a control interface 1858 and a display interface 1856 coupled to the display 1854. The display 1854 can be, for example, a TFT (Thin-Film-Transistor Liquid Crystal Display) display or an OLED (Organic Light Emitting Diode) display, or other appropriate display technology. The display interface 1856 can comprise appropriate circuitry for driving the display 1854 to present graphical and other information to a user. The control interface 1858 can receive commands from a user and convert them for submission to the processor 1852. In addition, an external interface 1862 can provide communication with the processor 1852, so as to enable near area communication of the mobile computing device 1850 with other devices. The external interface 1862 can provide, for example, for wired communication in some implementations, or for wireless communication in other implementations, and multiple interfaces can also be used.

The memory 1864 stores information within the mobile computing device 1850. The memory 1864 can be implemented as one or more of a computer-readable medium or media, a volatile memory unit or units, or a non-volatile memory unit or units. An expansion memory 1874 can also be provided and connected to the mobile computing device 1850 through an expansion interface 1872, which can include, for example, a SIMM (Single In Line Memory Module) card interface. The expansion memory 1874 can provide extra storage space for the mobile computing device 1850, or can also store applications or other information for the mobile computing device 1850. Specifically, the expansion memory 1874 can include instructions to carry out or supplement the processes described above, and can include secure information also. Thus, for example, the expansion memory 1874 can be provide as a security module for the mobile computing device 1850, and can be programmed with instructions that permit secure use of the mobile computing device 1850. In addition, secure applications can be provided via the SIMM cards, along with additional information, such as placing identifying information on the SIMM card in a non-hackable manner.

The memory can include, for example, flash memory and/or NVRAM memory (non-volatile random access memory), as discussed below. In some implementations, a computer program product is tangibly embodied in an information carrier. The computer program product contains instructions that, when executed, perform one or more methods, such as those described above. The computer program product can be a computer- or machine-readable medium, such as the memory 1864, the expansion memory 1874, or memory on the processor 1852. In some implementations, the computer program product can be received in a propagated signal, for example, over the transceiver 1868 or the external interface 1862.

The mobile computing device 1850 can communicate wirelessly through the communication interface 1866, which can include digital signal processing circuitry where necessary. The communication interface 1866 can provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication can occur, for example, through the transceiver 1868 using a radio-frequency. In addition, short-range communication can occur, such as using a Bluetooth, WiFi, or other such transceiver (not shown). In addition, a GPS (Global Positioning System) receiver module 1870 can provide additional navigation- and location-related wireless data to the mobile computing device 1850, which can be used as appropriate by applications running on the mobile computing device 1850.

The mobile computing device 1850 can also communicate audibly using an audio codec 1860, which can receive spoken information from a user and convert it to usable digital information. The audio codec 1860 can likewise generate audible sound for a user, such as through a speaker, e.g., in a handset of the mobile computing device 1850. Such sound can include sound from voice telephone calls, can include recorded sound (e.g., voice messages, music files, etc.) and can also include sound generated by applications operating on the mobile computing device 1850.

The mobile computing device 1850 can be implemented in a number of different forms, as shown in the figure. For example, it can be implemented as a cellular telephone 1880. It can also be implemented as part of a smart-phone 1882, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

Unless otherwise defined herein, scientific and technical terms used in this application shall have the meanings that are commonly understood by those of ordinary skill in the art. In case of conflict, the present specification, including definitions, will control.

Throughout this specification and embodiments, the word “comprise,” or variations such as “comprises” or “comprising,” will be understood to imply the inclusion of a stated integer or group of integers but not the exclusion of any other integer or group of integers. The term “including” or “includes” is used to mean “including but not limited to.” “Including” and “including but not limited to” are used interchangeably.

Any example(s) following the term “e.g.” or “for example” is not meant to be exhaustive or limiting. Unless otherwise required by context, singular terms shall include pluralities and plural terms shall include the singular. The articles “a,” “an” and “the” are used herein to refer to one or to more than one (e.g., to at least one) of the grammatical object of the article. All ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g., 1 to 6.1, and ending with a maximum value of 10 or less, e.g., 5.5 to 10. Each embodiment of this disclosure may be taken alone or in combination with one or more other embodiments of this disclosure.

In order for the disclosure to be more readily understood, certain terms are first defined. These definitions should be read in light of the remainder of the disclosure as understood by a person of ordinary skill in the art. Additional definitions are set forth throughout the detailed description. As referred to herein, the term “agricultural product” refers to any product produced by a plant or animal. Examples of agricultural products include, but are not limited to, fruits, vegetables, seeds, flowers, tubers, and bulbs. Specific examples of agricultural products include, but are not limited to, eggs, avocado, pomegranate, persimmon, apple, pear, citrus fruit, papaya, cherry, melon, guava, grape, mango, strawberries, raspberries, blueberries, grapefruit, stone fruit, and tomatoes. As referred to herein, the term “infection” refers to any pathogenic infection of an agricultural product. In some embodiments, an agricultural product infection can be a latent infection such that the infected agricultural product presents no visible symptoms of infection. In another embodiment, an agricultural product infection can manifest with visible symptoms, including, but not limited to, stem end rot, mold and/or vascular/internal browning.

Agricultural product infections can be caused by any pathogen and can include, for example, bacterial infections, viral infections, fungal infections, oomycete infections, or any combination thereof. In some embodiments, the infection is a fungal infection. Fungal infections can be caused by a pathogen belonging to one of the genera Colletotrichum (e.g., C. gloeosporioides, C. acutatum), Dothiorella (e.g., D. iberica, D. gregaria, D. aromatica), Neofusicoccum (e.g., N. luteum, N. parvum, N. australe), Diaporthe (e.g., D. neotheicola, D. cinnamomi), Lasiodiplodia (e.g., L. pseudotheobromae, L. theobromas), and Diplodia (e.g., D. mutila, D. pseuodoseriata, D. seriata), and belonging to the family Botryosphaeria, (e.g., B. dothidea). In alternative embodiments, the agricultural product infection can be caused by any other pathogen.

As used herein, the term “volatile compounds” refers to the compounds that are produced and emitted or outgassed from an agricultural product at ambient temperatures. Volatile compounds that are produced by agricultural products include, but are not limited to terpenoids, aldehydes, alcohols, acetates, and hydrocarbons. As used herein, the term “volatile compound profile” refers to a profile that represents one or more of the volatile compounds that are produced and emitted from an agricultural product at ambient temperature. As used herein, the term “volatile marker profile” refers to a profile based on one or more of the volatile compounds that are representative or characteristic of a certain trait and/or property of an agricultural product. Volatile marker profiles can characterize quality, predict infection, and predict or evaluate the ripening stage (under ripe, ripe, overripe), and/or flavor characteristics (e.g., sweet, bitter, juicy, dry, tart, and the like) of agricultural products. As used herein, the phrase “volatile ripening marker timeline” refers to a timeline based on a plurality of volatile marker profiles that characterize different stages of ripening of an agricultural product. The stages of ripening can include unripe, ripe and overripe stages of development, or any intermediate stages thereof. In some embodiments, the volatile ripening marker timeline comprises n stages of ripening, wherein n is an integer greater than 1, e.g., 2, 3, 4, 5, 6, 7, 8, 9, 10, etc.

As used herein, the term “flavor characteristics” refers to the taste and/or odor attributes of an agricultural product. The flavor characteristics include, but are not limited to, how sweet, bitter, tart, juicy, alcoholic, pungent, green, grassy, fruity, citrusy, leafy, terpy and/or woody an agricultural product is. As used herein, the term “ripe” refers to the time when an agricultural product is more preferred for human consumption. As used herein, the term “causative agent” refers to an organism (e.g., bacteria, virus, fungus or any other pathogen) that is responsible for the development of an infection or a disease. As used herein, the term “treatment” means to reduce, slow, prevent or inhibit the progression of an infection. As used herein, the term “treatment” may also refer to controlling and/or modifying the ripening rate of agricultural products. As used herein, the term “ethylene treatment” with regard to agricultural products refers to the application or use of any product which alters or aims to alter the ethylene composition, consumption, and/or detection in post-harvest products. In some implementations, an ethylene treatment can include the application of exogenous, gaseous ethylene to post-harvest products to alter and/or control the ripening rate of the agricultural products. In additional implementations, an ethylene treatment can include the use or application of an ethylene inhibitor, blocker, or absorber (e.g., 1-methylcycloprene (1-MCP)) to post-harvest plant products to alter and/or control ripening rate and/or to prolong shelf-life of the plant products.

While this specification contains many specific implementation details, these should not be construed as limitations on the scope of the disclosed technology or of what may be claimed, but rather as descriptions of features that may be specific to particular embodiments of particular disclosed technologies. Certain features that are described in this specification in the context of separate embodiments can also be implemented in combination in a single embodiment in part or in whole. Conversely, various features that are described in the context of a single embodiment can also be implemented in multiple embodiments separately or in any suitable subcombination. Moreover, although features may be described herein as acting in certain combinations and/or initially claimed as such, one or more features from a claimed combination can in some cases be excised from the combination, and the claimed combination may be directed to a subcombination or variation of a subcombination. Similarly, while operations may be described in a particular order, this should not be understood as requiring that such operations be performed in the particular order or in sequential order, or that all operations be performed, to achieve desirable results. Particular embodiments of the subject matter have been described. Other embodiments are within the scope of the following claims.

What is claimed is:

1. A system for determining quality characteristics of food items, the system comprising:
a tube configured to be placed in an environment containing one or more food items, the tube defining a fluid passageway;
a sorbent material positioned within the fluid passageway defined by the tube, wherein the tube with the sorbent material is configured to be positioned near one or more food items to collect one or more volatiles that are outgassed by the one or more food items and to be processed by a gas chromatograph machine, wherein the gas chromatograph machine is configured to receive the tube with the sorbent material after the volatiles collected on the sorbent material and generates output data that indicates presence and concentrations of the one or more volatiles desorbed from the sorbent material; and
a computing system configured to receive the output data generated by the gas chromatograph machine and determine, based on applying a machine learning model to the output data, a quality characteristic of the one or more food items, wherein the machine learning model was trained using (i) human observations of one or more quality characteristics of other food items, (ii) historic supply chain information of the other food items, and (iii) processed volatiles data associated with the other food items, wherein the other food items are a same type as the one or more food items.

2. The system of claim 1, wherein the sorbent material comprises 0.07 g to 0.14 g of a porous polymer.

3. The system of claim 1, further comprising:
an enclosure containing the one or more food items, wherein the enclosure defines a first aperture that provides fluid communication between gas contained within the enclosure and an ambient environment outside of the enclosure,
wherein the tube is configured to be positioned within the first aperture and, when positioned in the aperture, to provide a fluid passageway between the gas contained in the enclosure and the ambient environment.

4. The system of claim 3, wherein the enclosure defines a second aperture between the ambient environment and gas contained within the enclosure,
the system further comprising:
one or more fans that are in fluid communication with the second aperture and that are configured to provide a positive pressure flow of gas from the ambient environment into the enclosure that directs the volatiles outgassed by the one or more food items towards and through the tube containing the sorbent material.

5. The system of claim 3, wherein the enclosure defines a second aperture between the ambient environment and gas contained within the enclosure,
the system further comprising:
a pressurized gas supply that is in fluid communication with the second aperture and that is configured to provide a positive pressure flow of gas from the pressurized gas supply into the enclosure that directs the volatiles outgassed by the one or more food items towards and through the tube containing the sorbent material.

6. The system of claim 4, wherein the gas is at least one of ambient air and nitrogen.

7. The system of claim 3, wherein the enclosure is partially open on one or more sides of the enclosure.

8. The system of claim 1, wherein the quality characteristic includes a ripeness stage, stem rot, desiccation, taste, internal rot, mold, and firmness.

9. The system of claim 1, wherein the one or more food items comprise avocados, and the volatiles outgassed by the avocados include one or more of: aldehydes, alcohol, and terpenes.

10. The system of claim 1, wherein the one or more food items comprise mandarins, and the volatiles outgassed by the mandarins include one or more of: ethanol, ethyl acetate, ethyl-esters, acetaldehyde, alpha-pinene, limonene, linalool, germacrene D, and beta-farnesene.

11. The system of claim 1, wherein the historic supply chain information includes at least one of a place of origin of the other food items, a storage temperature of the other food items, shipping conditions associated with the other food items, and historic ripening information associated with the other food items.

12. The system of claim 1, wherein determining the quality characteristic of the one or more food items includes mapping the volatiles in the output data to one or more quality features that are identified from (i)-(iii).

13. The system of claim 1, wherein the computing system is further configured to:
identify supply chain information for the one or more food items that includes a preexisting supply chain schedule and destination for the one or more food items;
determine whether to modify the supply chain information for the one or more food item based on the determined quality characteristic of the one or more food items; and
in response to a determination to modify the supply chain information, generate modified supply chain information based on the determined quality characteristic, wherein the modified supply chain information includes one or more of a modified supply chain schedule and modified destination for the one or more food items.

14. A method for generating a trained model to determine a quality characteristic of a food item, the method comprising:
receiving, by a computing system, (i) volatiles data of a first plurality of food items that have a quality characteristic, (ii) volatiles data of a second plurality of food items that do not have the quality characteristic, (iii) human observations of other food items, and (iv) historic supply chain information associated with the other food items, wherein the first plurality of food items, the second plurality of food items, and the other food items are a same food type;
generating, by the computing system, a volatiles marker profile based on performing random forest modeling on (i) and (ii), wherein the volatiles marker profile indicates one or more volatiles whose concentrations are present in the first plurality of food items but not in the second plurality of food items; and
generating, by the computing system, a machine learning model based on mapping the volatiles marker profile to quality characteristics identified by (iii) and (iv), wherein the machine learning model correlates presence and concentrations of the volatiles with one or more quality characteristics of food items of the same food type.

15. The method of claim 14, wherein the volatiles data is non-destructively captured from the first and second plurality of food items.

16. A method for producing a volatile ripening marker timeline associated with agricultural products, the method comprising:
separating, by a computing system, a plurality of an agricultural product into n groups, wherein each group of the n groups represents a different ripeness stage of the agricultural product and n is an integer greater than 1;
independently assessing, by the computing system, presence and concentrations of one or more volatile compounds that are outgassed from each group of the n groups;
producing, by the computing system, a volatile marker profile for the agricultural product, wherein the volatile marker profile indicates the one or more volatile compounds whose presence and concentrations are identified in some but not all of the n groups; and
generating, by the computing system, a volatile ripening marker timeline, wherein each ripening stage represented in the volatile ripening marker timeline is correlated with the one or more volatile compounds in the volatile marker profile.

17. The method of claim 16, further comprising assessing, by the computing system, presence and concentrations of volatile compounds outgassed from an agricultural product based on comparing the presence and concentrations of the volatile compounds outgassed from the agricultural product to the volatile marker profile in the volatile ripening marker timeline.

18. The method of claim 17, further comprising predicting, by the computing system, a current ripeness stage of the agricultural product based on the volatile ripening marker timeline.

19. The method of claim 18, further comprising controlling, by the computing system, a ripening process of the agricultural product based on the predicted current ripeness stage of the agricultural product.

20. The method of claim 16, wherein the volatile ripening marker timeline includes a plurality of volatile marker profiles associated with each of a plurality of n stages of ripeness of the agricultural product, wherein each of the plurality of volatile marker profiles represent one or more volatile compounds whose presence and concentrations indicate a stage of ripeness as compared to other stages of ripeness, wherein n is an integer greater than 1.
